(19) United States
(12) Patent Application Publication
(10) Pub. No.: US 2025/0352530 A1
(43) Pub. Date: Nov. 20, 2025

(54) METHOD AND COMPOSITION FOR TREATING LUNG DISEASES

(71) Applicant: MannKind Corporation, Danbury, CT (US)

(72) Inventors: Wassim H. Fares, Lexington, MA (US); John J. Freeman, JR., New Fairfield, CT (US); Michael Castagna, Danbury, CT (US)

(21) Appl. No.: 19/210,846

(22) Filed: May 16, 2025

Related U.S. Application Data
(60) Provisional application No. 63/649,256, filed on May 17, 2024.

(51) Int. Cl. A61K 31/444; A61K 9/00; A61K 9/16; A61K 31/496

(52) U.S. Cl. CPC A61K 31/444; A61K 9/0075; A61K 9/1617; A61K 31/496

(57) ABSTRACT

A method, composition and kit for the treatment of interstitial lung disease, including fibrotic lung disease, are disclosed. The method utilizes a combination product for inhalation comprising a therapeutic amount of a dry powder formulation provided in an inhaler to be administered to a subject in need by oral inhalation. The composition comprises diketopiperazine particles and a kinase inhibitor for oral inhalation.

### CROSS-REFERENCE TO RELATED APPLICATIONS

This application claims the benefit of U.S. Provisional Patent Application No. 63/649,256, filed May 17, 2024, the entirety of which is incorporated herein.

### TECHNICAL FIELD

The present disclosure relates to methods, compositions, and kits for therapeutic treatment of lung diseases or disorders, including interstitial lung diseases such as idiopathic pulmonary fibrosis. In particular, the methods, compositions and kits comprise a combination product comprising a dry powder inhalation and methods of treating lung diseases by oral inhalation.

### BACKGROUND

Idiopathic pulmonary fibrosis (IPF) is a chronic lung disease of yet unknown causes and there is no cure for IPF. The disease is progressive and irreversible and causes scar tissue (fibrosis) to build up in the lungs, which makes the lungs unable to transport oxygen into the bloodstream effectively. It affects people between the ages of 50 and 70. It belongs to a group of conditions called interstitial lung diseases (ILD), which describes lung diseases that involve inflammation or scarring in the lung. The most common signs and symptoms of IPF are shortness of breath and a persistent dry, hacking cough. Subjects affected with IPF also experience a loss of appetite and gradual weight loss. In individuals with IPF, scarring of the lungs increases over time until the lungs can no longer provide enough oxygen to the body's organs and tissues.

Currently, there are no procedures, or medications that can remove the progressive scarring of lung tissue. Therefore, it is important to learn good coping skills and educate the patient about the disease. Generally, treatments are designed to slow progression of scar formation in the lungs, and these may not necessarily lessen the symptoms of cough and breathlessness associated with the disease. Oral tablet forms of pirfenidone and nintedanib therapies have been shown to slow the progression of IPF; however, some patients cannot tolerate these medications at the dosage needed to slow down progression due to the side effects. With repeated and necessary high dosing to slow disease progression, there are too many adverse effects, including gastrointestinal such as nausea, diarrhea, abdominal pain, vomiting; hepatobiliary, nervous system, vascular, metabolism and nutritional disorders.

There are some additional medications that are useful to improve the symptoms of IPF, including shortness of breath and cough. Some of these medications include, for example, anti-acids to prevent gastroesophageal reflux and opioids to treat shortness of breath. Oxygen therapy and exercise training to increase oxygen levels are recommended to subjects with IPF, as well as education and support for people with chronic condition in order to provide them with pulmonary rehabilitation. Moreover, one major and invasive treatment is to provide the patient with lung transplant. Therefore, there is a need to improve or provide a patient with IPF alternate and new methods of treatment to treat the disease.

Drug delivery to lung tissue is accomplished using a variety of methods and routes of administration. For example, oral drug delivery, or enterally, such as tablets and capsules containing the medication, and parenterally, including, injections of targeted drugs to treat the disease or symptoms of the disease. Devices for inhalation, including nebulizers and inhalers, such as metered dose inhalers and dry powder inhalers are also used to treat local respiratory tract or lung disease or disorders.

Some dry powder inhaler products developed for pulmonary delivery have met with success to date. However, due to lack of practicality for use, and/or cost of manufacture, there is room for improvement. Some of the persistent problems observed with prior art inhalers include, lack of device ruggedness, inconsistency in dosing, inconvenience of the equipment, and poor deagglomeration of the powders. With some devices, the need to use harmful propellants to deliver a dose has limited therapy, and high manufacturing costs, and/or lack of patient compliance discourages their production. In addition, delivering the active ingredient directly to the target organ can decrease the dose and can cause less side effects than by other routes of administration. Therefore, the inventors have identified the need to design and manufacture new formulations and inhalers, which will provide consistent, or improved powder delivery properties, are easy to use, and have discrete configurations which would allow for better patient compliance.

### SUMMARY

Disclosed herein are methods and compositions for the treatment of lung diseases and/or disorders, including, interstitial lung disease, for example, idiopathic pulmonary fibrosis (IPF), progressive pulmonary fibrosis, and scleroderma. In embodiments herewith, a dry powder composition is provided in a dry powder inhaler, for example, a single dose dry powder inhaler, or a multiple dose dry powder inhaler comprising a replaceable cartridge, or capsule. In one embodiment, a dry powder pharmaceutical formulation for inhalation is provided for delivery to the lungs for local or systemic delivery into the pulmonary circulation. The pharmaceutical formulation comprises a dry powder for inhalation comprising a protein kinase inhibitor, for example, a small organic molecule that inhibits the functioning of protein kinases and diketopiperazine particles for lung delivery. A dry powder inhaler is also provided, which is a single use inhaler, or multiple use breath-powered inhaler, compact, reusable, or disposable for use for the effective and rapid delivery of powder medicament to the lungs and the systemic circulation of a subject newly diagnosed with interstitial lung disease, or an individual suffering with a chronic and progressive fibrotic disease type.

In one embodiment, a method of treating idiopathic pulmonary fibrosis comprises providing a drug delivery system, which is designed for drug delivery to the lungs by oral inhalation, and administering the active agent in a therapeutically effective dose of a pharmaceutical composition comprising the active agent, including nintedanib or an indolinone, salt thereof, an ester thereof or a nintedanib derivative thereof for rapid delivery and onset of action of the active agent being delivered to lung tissue and that the active agent reaches the alveoli and the systemic circulation in the lungs. In the method, the active agent molecule can reach its target site in a therapeutically effective manner and with less adverse effects. In one embodiment, the method of treatment comprises treating or administering to a patient diagnosed with a lung disease or disorder, in particular, fibrotic and/or inflammatory disease of the lungs, including, idiopathic lung disease, for example, idiopathic pulmonary fibrosis and in need of treatment, a therapeutic dose of a dry powder formulation comprising one or more kinase inhibitors for treating the disease. In one embodiment, the dose of the dry powder is delivered to the lungs using a dry powder inhaler, and wherein the kinase inhibitor can reach the deep lung. In one embodiment, the pharmaceutical composition is self-administered by the patient with one or more breaths using a breath-powered dry powder inhaler for oral or nasal inhalation. The delivery system can reduce the adverse effects caused by oral tablets or capsule, including gastrointestinal side effects such as nausea, diarrhea, abdominal pain, vomiting; hepatobiliary, nervous system, vascular, metabolism and nutritional disorders,

In one embodiment, the method further comprises administering to a subject in need of treatment a stable pharmaceutical composition comprising, one or more active agents, for delivery to lung tissue, wherein more than one active agent can be formulated together or formulated separately to be administered separately and at different intervals during a therapy. In another embodiment, the pharmaceutical composition comprises a formulation for inhalation comprising a therapeutically effective dose of a dry powder comprising one or more active agents, including, a small molecule, for example, nintedanib, imatinib, pirfenidone, analogs thereof, and/or derivatives thereof, including prodrugs, which inhibit the mechanisms of scar formation in the lungs of a patient treated for such condition.

In an exemplary embodiment, a dry powder formulation for inhalation is provided comprising a small molecule, including inhibitors of scar formation in the lungs for treating fibrotic disease. In an embodiment, a kinase inhibitor prevents scaring or an inflammatory cascade reaction by binding to the membrane receptors with kinase activity on the surface of cells, which results in inhibition of scar formation in lung tissue. In one embodiment, a dry powder formulation is provided comprising a diketopiperazine and a kinase inhibitor compound, which is targeted against key protein kinases of cells to inhibit phosphorylation of certain cellular signaling pathways that regulate abnormal gene expression and cause fibrotic disease in particular in the lungs. In embodiments, a kinase inhibitor compound is targeted against kinase molecules, including kinases that transfer a γ-phosphate group from adenosine triphosphate (ATP) to serine, threonine, or tyrosine amino acid residues. In another embodiment, the pharmaceutical compositions for treating lung disease comprise kinase inhibitors, which are classified as type I inhibitors.

In one embodiment, the inhalable pharmaceutical composition can comprise one or more pharmaceutically acceptable carrier and/or excipient, which is a surfactant, an amino acid, and/or a phospholipid, or combinations thereof.

In another embodiment, the inhalable pharmaceutical composition for treating ILD, including IPF comprises one or more active agents and a diketopiperazine having the formula:

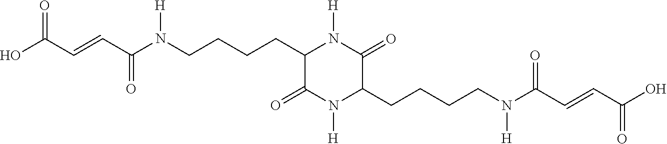 [SMILES: O=C(O)/C=C/C(=O)NCCCCC1NC(=O)C(CCCCNC(=O)/C=C/C(=O)O)NC1=O]

wherein the diketopiperazine is provided in an amorphous powder, in a crystalline form, or in a microcrystalline particle form, or combinations thereof. In one embodiment, the inhalable pharmaceutical composition is in a crystalline dry powder comprising a therapeutically effective dose of Compound I having the formula:

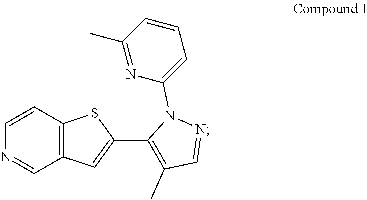 [SMILES: Cc1cccc(-n2ncc(C)c2-c2cc3cnccc3s2)n1]

wherein the Compound I content in a dose of the formulation ranges from about 1 mg to about 100 mg, or up to about 150 mg (w/w) in the dry powder composition, and wherein the dose is administered once or more times a day. In another embodiment, the dose can comprise Compound I content in a therapeutically dose can comprise from about 0.5 mg to about 9 mg, from about 1 mg to about 7.5 mg, from about 15 mg to about 30 mg, from about 30 mg to about 50 mg, from about 20 mg to about 60 mg, or from about 1 mg to about 20 mg.

In some embodiments, the inhalable pharmaceutical composition comprises a dry powder comprising one or more pharmaceutically acceptable carrier and/or excipients selected from lactose, mannose, sucrose, mannitol, trehalose, sodium citrate, trisodium citrate, zinc citrate, glycine, L-leucine, isoleucine, trileucine, sodium tartrate, zinc tartrate, in methionine, vitamin A, vitamin E, sodium chloride, zinc chloride, microcrystalline cellulose, polyvinylpyrrolidone and polysorbate 80, or combinations thereof.

In other embodiments, the inhalable pharmaceutical composition comprises a dry powder comprising one or more pharmaceutically acceptable carriers and/or excipients selected from the group consisting of sodium citrate, sodium chloride, leucine or isoleucine and trehalose, or combinations thereof.

In certain embodiments, the inhalable pharmaceutical composition comprises microcrystalline particles of 3,6-bis(N-fumaryl-4-aminobutyl)-2,5-diketopiperazine which have a specific surface area ranging from about 20 m²/g to about 63 m²/g, from about 10 m²/g to about 35 m²/g; from about 15 m²/g to about 30 m²/g. In one embodiment, the microcrystalline particles have a pore size ranging from about 23 nm to about 30 nm.

In some embodiments, a method of treating interstitial lung disease, including, idiopathic pulmonary fibrosis comprises administering to a patient in need of treatment by oral inhalation a dry powder composition comprising diketopiperazine particles and from 0.1 mg to about 0.9 mg, 1 mg to 5 mg, from 5 mg to 10 mg; 10 mg to 15 mg, from about 15 to about 20 mg; 20 mg to 30 mg, 30 mg to 50 mg; 50 mg to 100 mg; 100 to 150 mg; or 150 to 300 mg per inhalation session of a Compound I, a pharmaceutically acceptable salt thereof, a derivative thereof, and, optionally, a pharmaceutically acceptable carrier and/or excipient, wherein the dry powder composition is provided in a dry powder inhaler in single dose cartridges. In one embodiment, multiple cartridges can be provided to the patient for a predetermined dose depending on the patient's need.

In embodiments herewith, wherein the method comprises pirfenidone, the patient is administered a therapeutically effective dose of the dry powder composition is provided to the patient separately, in a blister, or pouch having one or more capsules or cartridges for adapting to a dry powder inhaler prior use, wherein each capsule or cartridge comprises up to 30 mg, or 50 mg of the compound. In one embodiment, the therapeutically effective dose per day can comprise up to 500 mg; up to 750 mg; up to 1,000 mg, or up to 2,500 mg wt % of the compound per day, which is provided in multiple cartridges for inhalation with a dry powder inhaler. The administration can be carried out in one or more dosing sessions.

In this and other aspects, the method utilizes a composition comprising, one or more pharmaceutically acceptable carrier and/or excipients, which is selected from the group consisting of fumaryl diketopiperazine, lactose, mannose, sucrose, mannitol, trehalose, sodium citrate, trisodium citrate, zinc citrate, glycine, L-leucine, isoleucine, trileucine, sodium tartrate, zinc tartrate, methionine, vitamin A, vitamin E, sodium chloride, zinc chloride, polyvinylpyrrolidone, and a surfactant such as polysorbate 80.

In alternate embodiments, the method for treating interstitial lung disease, including idiopathic pulmonary fibrosis comprises administering to a subject in need of treatment a pharmaceutically effective amount of a dry powder comprising Compound I of the formula 2-[4-methyl-1-(6-methylpyridin-2-yl)-1H-pyrazol-5-yl]thieno-[3,2-c]pyridine, a pharmaceutically acceptable salt thereof, an analog thereof, and/or prodrug thereof, wherein the one or more pharmaceutically acceptable carrier and/or excipient are sodium citrate, sodium chloride, leucine or isoleucine, or trehalose.

In one embodiment, a method of treating pulmonary fibrosis comprises, administering to a patient in need of treatment, an inhalable dry powder pharmaceutical composition comprising: a diketopiperazine and Compound I, optionally, in combination with Compound II having the formula:

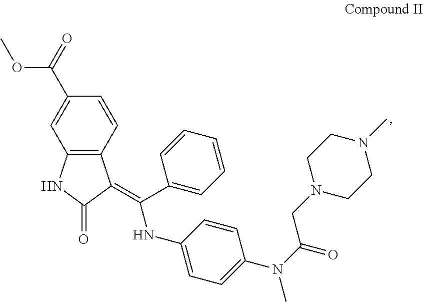

or methyl 2-hydroxy-3-[N-[4-[methyl-[2-(4-methylpiperazin-1-yl) acetyl]amino]phenyl]-C-phenylcarbonimidoyl]-1H-indole-6-carboxylate, a pharmaceutically acceptable salt thereof including an esylate, or analogs thereof, and optionally, one or more pharmaceutically acceptable carriers and/or excipients; wherein the diketopiperazine is in an amorphous form, in a crystalline form, or in a crystalline composite particle form, or combinations thereof, and the diketopiperazine has the formula:

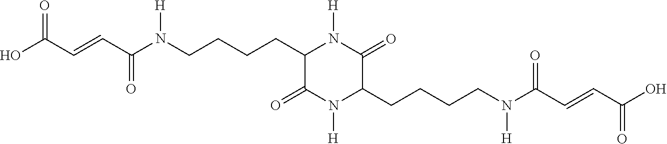 [SMILES: O=C(O)/C=C/C(=O)NCCCCC1NC(=O)C(CCCCNC(=O)/C=C/C(=O)O)NC1=O]

In some embodiments, the method of treating interstitial lung disease and in particular, idiopathic pulmonary fibrosis comprises, administering to a patient in need of treatment and inhalable pharmaceutical dry powder comprising Compound I, or Compound II (nintedanib) by oral inhalation using a dry powder inhaler comprising a movable member for mounting a cartridge, or capsule comprising a dose of the dry powder and having a container, which can attain a dosing configuration upon being loaded onto the inhaler, wherein said cartridge comprises the dry powder composition to be inhaled. In one embodiment, the dry powder inhaler cartridge consisting of a lid and a container and a dry powder dose that is provided separately prior to use. In one embodiment, nintedanib or another kinase inhibitor provided to a patient is in amounts per dosing of about 0.2 mg to about 30 mg, or from about 0.1 to about 0.4 mg, or from about 0.5 mg to about 4 mg, 0.5 mg, 1 mg, 3 mg, 5 mg, 7 mg, 8 mg, 9 mg, 10 mg, 12 mg, 15 mg, 20 mg of powder comprising from 1% to about 40% (w/w), from about 5% to 10%, from about 10% to about 20%, from 20 to 30 or from 30% to about 40% or more. In some embodiments, the amount of a kinase inhibitor in the dry powder is from about, or about 5%, 10%, 15%, 20%, 25%, 30%, 35% or 40% (w/w) nintedanib in the composition. In some embodiments, Compound I or nintedanib is provided as 10 mg of a 20% w/w dry powder composition in a cartridge/container, thereby providing a 2 mg dose of Compound I or nintedanib. In another embodiment, the amount of Compound I or nintedanib to be administered to a patient comprises one of more cartridges containing the dry powder composition of nintedanib per dose session and wherein the disease is pulmonary fibrosis.

In another embodiment, the dry powder comprising nintedanib or other kinase inhibitor compound are stable at room temperature (25° C./60% relative humidity) for a period of at least 1 year. In this and other embodiments, the kinase inhibitor dry powder composition can be stored at room temperature for up to 1 year, 2 years, 3 years or longer. In this embodiment, the dry powder comprising nintedanib can be stored in a blister package. The dry powder is also stable at higher temperatures, for example, for use in warm climates due to its stability, for example, it is stable up to about 10 to 12 weeks at a temperature of 40° C. and 70% relative humidity. In one embodiment, the dry powder comprises a kinase inhibitor, for example, Compound I or nintedanib can comprise from about 1 wt % to about 65 wt %, from 1 wt % to about 60 wt %, or from about 25 wt % to about 60 wt % in the composition.

In another embodiment, the method of treating IPF comprises providing a patient in need of treatment an inhaler and one or more cartridges comprising a dose of a dry powder composition and having the patient inhale the one or more cartridges contents from each of the one or more cartridges, wherein the one or more cartridges can deliver an effective dose of up to 300 mg pre dosing session of Compound I or Compound II of the formula:

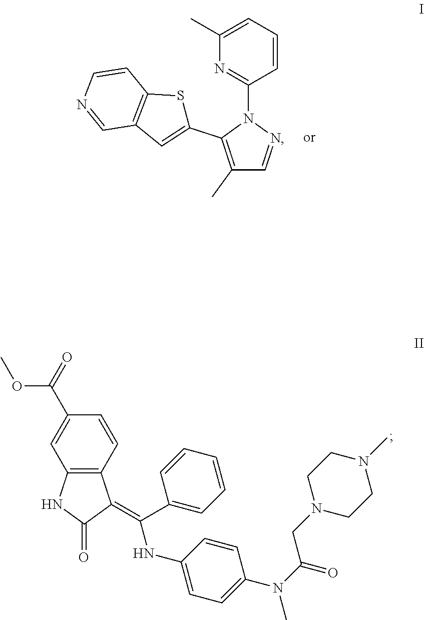

or pharmaceutically acceptable salts thereof, or esters and/or analogs thereof, and wherein the dry powder composition comprises particles of a pharmaceutically acceptable excipient having the formula 3,6-bis-(N-fumaryl-4-aminobutyl)-2,5-diketopiperazine. In one embodiment, the method comprises having the patient inhale for at least 4 to 10 seconds, or 2 to 6 seconds per inhalation using a high resistance dry powder inhaler having a resistance value from about 0.05 to about 0.200 (kPa)/liter/min. In one embodiment, the pharmaceutical composition for the treatment of pulmonary disease can comprise a dose of nintedanib of from about 0.01 mg to about 2 mg per dose, from about 1 mg to about 5 mg, from about 5.5 mg to about 10 mg, from about 10 mg to about 20 mg, from about 20 mg to about 40 mg, from about 40 mg to about 60 mg, from about 60 mg to about 80 mg, from about 80 mg to about 100 mg, or from about 100 mg to about 150 mg per day, which can be administered in one session, two sessions, three sessions, or more.

In some embodiments, the method of treatment of interstitial lung disease, including, pulmonary fibrosis, progressive pulmonary fibrosis, or scleroderma comprises, administering to a subject in need of treatment, a pharmaceutical composition comprising Compound I and/or nintedanib (Compound II) separately, sequentially or combinations thereof with one or more of a vasodilator compound, including treprostinil. In one embodiment, the method comprises a combination therapy comprising, administering to the subject a vasodilator comprising one or more of: sildenafil, tadalafil, vardenafil, a prostaglandin, a prodrug thereof, a prostaglandin derivative, a prostaglandin analog, for example, treprostinil, or a pharmaceutically acceptable salt of these compounds thereof, including, treprostinil sodium, or prodrugs thereof. In another embodiment, the method comprises treating interstitial lung disease and pulmonary arterial hypertension simultaneously by delivering to the lungs of the patient a combination therapy comprising a dry powder formulation comprising Compound I and/or nintedanib (Compound II) and/or a dry powder composition comprising a vasodilator compound, including, treprostinil, or a pharmaceutically acceptable salt of these compounds thereof, comprising one or more of: treprostinil sodium, or prodrugs thereof, and into the systemic circulation of a subject, by way of pulmonary inhalation using a dry powder inhaler.

In one embodiment, the method comprises providing to a patient in need of treatment a dry powder inhaler comprising the active agent, for example, Compound I, nintedanib (Compound II), pirfenidone, or treprostinil in a stable dry powder formulation, and administering the active agent by oral inhalation. In one embodiment, the vasodilator can be formulated together with the pirfenidone, nintedanib in the same formulation or separately and administered separately in its own formulation and provided to the patient at different intervals, or sequentially during a dosing session.

In one embodiment, the drug delivery system comprises a dry powder inhaler comprising a diketopiperazine-based drug formulation for delivering small molecules, for example, Compound I, pirfenidone, nintedanib (Compound II), a prostaglandin, or pharmaceutically acceptable salts thereof, prodrugs, or analogs thereof, including, tresprostinil and protein-based products for treating pulmonary fibrosis and PAH. The method provides advantages over typical methods of drug delivery, such as, oral tablet and subcutaneous and intravenous injectable/infusion drug products that are sensitive to degradation and/or enzymatic deactivation.

In certain embodiments disclosed herein, a method is provided for the treatment comprises further providing to a patient with pulmonary fibrosis and PAH a prostaglandin, treprostinil, or a pharmaceutically acceptable salt of these compounds thereof, including, treprostinil, treprostinil sodium, or prodrugs thereof or derivative thereof, in a dry powder formulation. The method comprises: selecting a patient to be treated for PAH and interstitial lung disease, and administering to the patient a dry powder formulation comprising: Compound I, nintedanib, pirfenidone, or treprostinil or a treprostinil salt or derivative thereof; wherein the treprostinil is combined with diketopiperazine microcrystalline particles to produce a pharmaceutical formulation, or composition suitable for pulmonary inhalation and, having the patient inhale from an inhaler containing the composition and delivering the treprostinil formulation using a breath-powered dry powder inhaler. In this and other embodiments, the dry powder formulation is provided in a reconfigurable cartridge comprising from about 1 μg to about 300 μg or more of treprostinil or a salt thereof in the dry powder formulation per dose. In certain embodiments, the dry powder formulation can comprise from about 10 μg to about 300 μg of treprostinil per dose in a cartridge or capsule. In one embodiment, a cartridge for single use can comprise from about 10 μg to about 90 μg of treprostinil for at least one inhalation. In some embodiments, the dry powder formulation is delivered using at least one inhalation per use. In this and other embodiments, the dry powder formulation is delivered to a patient in less than 10 seconds, less than 8 seconds, less than 6 seconds, or less than 4 seconds per inhalation or breath. In one embodiment, the pharmaceutical dry powder composition comprises microcrystalline particles of fumaryl diketopiperazine wherein the particles have a specific surface area ranging from about 59 m²/g to about 63 m²/g, or from about 35 m²/g to about 59 m²/g and have a pore size ranging from about 23 nm to about 30 nm.

Also disclosed herein is a method of treating a pulmonary fibrosis concomitant with pulmonary arterial hypertension disease or disorder comprising, selecting a patient to be treated with pulmonary arterial hypertension, or a patient with PAH, which exhibits a condition treatable with an active agent, including treprostinil, epoprostenol, bosentan, ambrisentan, macitentan, sildenafil, tadalafil, vernadafil, riociguat and the like, analogs thereof, or combinations thereof, which patients are treated only by oral or injectable administration, and replacing the aforementioned therapy with an inhalation therapy comprising providing the patient with an inhaler comprising the active agent in a stable dry powder composition for treating the disease or disorder; wherein the stable dry powder composition comprises the active agent and a diketopiperazine; and administering the stable dry powder composition to the patient by pulmonary inhalation; thereby treating the disease or condition.

In an embodiment, the formulation for treating pulmonary arterial hypertension and/or interstitial lung disease comprises treprostinil or a salt thereof, in an amount up to 300 μg per dose, for example, amounts of 1 μg, 5 μg, 10 μg, 15 μg, 20 μg, 30 μg, 60 μg, 90 μg, 100 μg, 120 μg, 150 μg, 180 μg, 200 μg, or 300 μg, and one or more pharmaceutically acceptable carriers and/or excipients per dose are to be administered to a subject. In this embodiment, the pharmaceutically acceptable carrier and/or excipient can be formulated for oral inhalation and can form particles, and may include one or more of a diketopiperazine, including, fumaryl diketopiperazine, sugars such as mannitol, xylitol, sorbitol, and trehalose; amino acids, including, glycine, leucine, isoleucine, methionine; surfactants, including polysorbate 80; cationic salts, including, monovalent, divalent and trivalent salts, including, sodium chloride, potassium chloride, magnesium chloride, and zinc chloride; buffers such as citrates and tartrates, or combination of one or more carriers and/or excipients and the like. In another embodiment, the formulation comprises a dry powder comprising treprostinil, a sugar and an amino acid, wherein the sugar is mannitol or trehalose; and the amino acid is leucine or isoleucine and a cationic salt. In certain embodiments, the formulation can further comprise sodium chloride, potassium chloride, magnesium chloride or zinc chloride, sodium citrate, sodium tartrate, or combinations thereof.

In an embodiment, a combination therapy comprises a method of treating pulmonary arterial hypertension and/or interstitial lung disease comprising: administering to a patient a dose of nintedanib, treprostinil, or combinations thereof. In these embodiments, the treprostinil dose and/or the nintedanib dose of the combination therapy is administered using a dry powder inhaler for oral inhalation. In some embodiments, the nintedanib dose and the treprostinil dose of the combination therapy are administered in the same dry powder inhaler provided with different cartridges/containers. In some embodiments, the nintedanib dose and the treprostinil dose are administered from different dry powder inhalers, each provided with its own cartridges/containers. In some embodiments, the combination therapy involves inhalation of the treprostinil dose and the nintedanib dose sequentially at about the same time, e.g., within about 0 to about 15 minutes of each other. In other embodiments, the combination therapy involves inhalation of the treprostinil dose and the nintedanib dose at different times. In other embodiments, the combination therapy involves inhalation of the treprostinil dose and the nintedanib dose about 30 minutes apart, about 1 hour apart, about 2 hours apart, about 3 hours to about 6 hours apart, about 6 hours to about 9 hours apart, or about 9 hours to about 12 hours apart. In some embodiments, the combination therapy involves one or more breaths of the nintedanib inhalable powder and/or one or more breaths of the treprostinil inhalable powder using the same or different dry powder inhalers.

In an embodiment, a treprostinil inhalation powder dose is provided to a patient suffering with pulmonary arterial hypertension and in need of treatment. In some embodiments, a dry powder inhaler comprises a container including a cartridge, and the container or the cartridge comprises the dry powder comprising treprostinil. In some embodiments, treprostinil is administered in multiple daily doses for a period of six months and the treprostinil is administered by oral inhalation at an earlier time in the course of the disease to patients with Functional Class II as a first line monotherapy.

In alternate embodiments, the dry powder for inhalation may be formulated with other carriers and/or excipients other than diketopiperazines, for example a sugar, including trehalose; buffers, including sodium citrate; salts, including sodium chloride and zinc chloride, and one or more active agents, including, treprostinil, vardenafil, and sildenafil.

In embodiments herewith, the method of treating interstitial lung disease in a patient also with PAH comprises, administering to a patient with moderate to severe PAH a dry powder formulation comprising, an active agent, including, treprostinil and a pharmaceutically acceptable carrier and/or excipient, including, a diketopiperazine, wherein the treprostinil in an amount up to 200 μg per dose per dosing session, and the formulation is administered using a dry powder inhaler one or more times daily.

In one embodiment, the dry powder inhaler comprises a movable member for loading a container comprising the pharmaceutical composition and the movable member can configure a container to attain a dosing configuration from a container loading configuration so that the inhaler creates an airflow through the inhaler during an inhalation maneuver to allow the contents of the container to enter the airflow path and greater than 60% of a dry powder dose in the container is delivered to the lungs in a single inhalation. In one embodiment, the method comprises administering a second dry powder composition comprising one or more aforementioned active agents.

In some embodiments, the treatment regimen with an inhalable dry powder depends on the patient's need and can be one inhalation to replace each of a nebulization session performed with standard therapy, including, at least one to four inhalations per day depending on the severity of disease. In some embodiments, the composition can be administered in one or more breaths per session depending on the dose requirement of the patient.

In an embodiment, the method of treatment interstitial lung disease such as IPF, PPF or scleroderma comprises, administering to a subject in need of treatment a therapeutically effective dose of inhalable nintedanib composition by oral inhalation alone for naïve patients using a dry powder inhaler, or in combination with one or more therapies administered by alternative routes of administration, including, oral capsules and tablets, in combination with oral doses such as 100 or 150 mg of oral capsules, Ofev®, for example, one or more times a day. In one embodiment, the method of treatment interstitial lung disease, comprises administering an inhalable nintedanib composition by oral inhalation using a dry powder inhaler in combination with pirfenidone administered as an oral tablet or capsule of about 267 mg, 534, or about 800 mg, daily or up to 3 times a day as needed or tolerated by the patient. In an embodiment, the method of treatment comprises administering to a patient in need of treatment for ILD, including, IPF, PPF and scleroderma, an inhalable dry powder composition comprising nintedanib and a diketopiperazine, including, 3,6-bis(N-fumaryl-4-aminobutyl)-2,5-diketopiperazine, wherein the nintedanib is in an amount of about of up to 35 mg in a single inhalation. In some embodiments, the inhalable composition comprises single doses of 2 mg, 4 mg, 8 mg, 10 mg, 12 mg, 14 mg, 16, mg, 18 mg, 20 mg, 22, mg, 24 mg of nintedanib administered one or more times daily, in particular, once, twice, thrice or up to 4 times daily.

In another embodiment, a method of reducing or slowing progression of IPF comprises administering to a patient in need of treatment a therapeutically effective amount of an inhalable dry powder composition comprising an indole-derived inhibitor of a multiple receptor tyrosine kinase and non-receptor tyrosine kinases in the lung of the patient using a dry powder inhaler and oral inhalation, which reduces adverse side effects of oral capsule or tablet therapy. In one embodiment, the patient to be treated is incapable of tolerating the dosage amount given by oral capsules or tablets of nintedanib due to adverse effects including, diarrhea, nausea, abdominal pain, and vomiting. In one aspect of this embodiment, it is contemplated that patients treated with the inhalable dry powder composition in combination with oral therapy can be weaned of the large oral doses of nintedanib, pirfenidone, or other tyrosine kinase inhibitors administered by oral tablets or capsule therapies.

### BRIEF DESCRIPTION OF DRAWINGS

FIG. 1 depicts a graphic representation of data obtained from room temperature stability studies collected from the present composition comprising T powders formulated with nintedanib at 20% content in the composition. Samples were assayed at various times after being incubated at 25° C., 60% RH for a period of a year.

FIG. 2 depicts a graphic representation of data obtained from room temperature stability studies collected from the present composition comprising nintedanib free base as control for samples in FIG. 1. Samples were assayed at various times after being incubated at 25° C., 60% RH for a period of a year.

FIG. 3 depicts a graphic representation of data obtained from room temperature stability studies collected from the present composition comprising T powders formulated with nintedanib at 20% content in the composition. Samples were assayed at various times after being incubated at 40° C., 75% RH for a period of 12 weeks.

FIG. 4 depicts a graphic representation of data obtained from room temperature stability studies collected from the present composition comprising nintedanib free base as control for samples in FIG. 1. Samples were assayed at various times after being incubated at 40° C., 75% RH for a period of 12 weeks.

FIG. 5 depicts a graphic representation of data obtained from pharmacokinetic studies performed in Sprague Dawley rats using an exemplary dry powder comprising Compound I are described herewith. The instant dry powder was administered by insufflation at circles on graph. For comparison, the data also shows samples from animals, which were administered Compound I via IV injection (squares).

FIG. 6 depicts a graphic representation of mean plasma profile data obtained from six month pharmacokinetic studies performed in beagle dogs using an exemplary nintedanib inhalation powder described herewith.

FIG. 7 depicts a graphic representation of data obtained from six month pharmacokinetic studies performed in beagle dogs using an exemplary nintedanib inhalation powder described herewith.

### DETAILED DESCRIPTION

In embodiments disclosed herein are methods of treating interstitial lung disease in particular, pulmonary fibrosis in patients with disease, including, fibrosis of the lungs. In one embodiment, the method comprises administering to a patient in need of treatment one or more dry powder compositions using dry powder inhalers, and delivering the dry powder compositions comprising Compound I, nintedanib, pirfenidone, and/or treprostinil, and salts, ester prodrugs and/or analogs thereof, to the respiratory tract and deep lung.

In an exemplary embodiment a dry powder delivery system comprises a dry powder inhaler for single use of a pharmaceutical dose in a container or a cartridge for delivering the dry powders, including the pharmaceutical medicaments to a subject by oral inhalation. In one embodiment, the dry powder inhaler is a breath-powered, dry powder inhaler, and the container or cartridge is designed to contain an inhalable dry powder, including, but not limited to pharmaceutical formulations comprising an active ingredient, including a pharmaceutically active substance, and optionally, one or more than one pharmaceutically acceptable carrier and/or excipients. In particular, the dry powder inhaler containing the pharmaceutical compositions are for the treatment of pulmonary fibrosis and/or pulmonary arterial hypertension.

The dry powder inhalers are provided in various embodiments of shapes and sizes, and can be reusable, easy to use, inexpensive to manufacture and/or produced in high volumes in simple steps using plastics or other acceptable materials. Various embodiments of the dry powder inhalers are provided herein and in general, the inhalation systems comprise inhalers, powder-filled cartridges, and empty cartridges. The present inhalation systems can be designed to be used with any type of dry powder. In one embodiment, the dry powder is a relatively cohesive powder which requires optimal deagglomeration conditions. In one embodiment, the inhalation system provides a re-useable, miniature breath-powered inhaler in combination with single-use cartridges containing pre-metered doses of a dry powder formulation. The inhaler can deliver a dry powder dose in a single inhalation per use in treating interstitial lung disease with or without pulmonary arterial hypertension, in less than 10 seconds, or less than 6 seconds or less than 4 seconds per cartridge session. In another embodiment, oral inhalation through the inhalers can deliver greater than 60% of a powder dose in less than 6 seconds, in less than 4 seconds and in less than 2 seconds.

As used herein the term “a unit dose inhaler” refers to an inhaler that is adapted to receive a single enclosure, cartridge or container comprising a dry powder formulation and delivers a single dose of a dry powder formulation by inhalation from a single container to a user. In some instances, multiple unit doses will be required to provide a user with a specified dosage and that the same inhaler can be used for multiple unit dose delivery and in multiple dose sessions for a predetermined number of use sessions.

As used herein a “cartridge” is an enclosure configured to hold or contain a dry powder formulation, a powder containing enclosure, which has a cup or container and a lid. The cartridge is made of rigid materials, and the cup or container is moveable relative to the lid in a translational motion or vice versa and can attain a closed configuration to hold a dry powder and a dosing configuration in use with an inhaler.

As used herein a “powder mass” is referred to an agglomeration of powder particles or agglomerate having irregular geometries such as width, diameter, and length.

As used herein a “unit dose” refers to a pre-metered dry powder formulation for inhalation. Alternatively, a unit dose can be a single enclosure including a container having a single dose or multiple doses of formulation that can be delivered by inhalation as metered single amounts. A unit dose enclosure/cartridge/container contains a single dose. Alternatively, it can comprise multiple individually accessible compartments, each containing a unit dose.

As used herein, the term “about” is used to indicate that a value includes the standard deviation of error for the device or method being employed to determine the value.

As used herein, the term “microparticle” refers to a particle with a diameter of about 0.5 to about 1000 μm, irrespective of the precise exterior or interior structure. Microparticles having a diameter of between about 0.5 and about 10 microns can reach the lungs, successfully passing most of the natural barriers. A diameter of less than about 10 microns is required to navigate the turn of the throat and a diameter of about 0.5 μm or greater is required to avoid being exhaled. To reach the deep lung (or alveolar region) where most efficient absorption is believed to occur, it is preferred to maximize the proportion of particles contained in the “respirable fraction” (RF), generally accepted to be those particles with an aerodynamic diameter of about 0.5 to about 6 μm, though some references use somewhat different ranges, as measured using standard techniques, for example, with an Anderson Cascade Impactor. Other impactors can be used to measure aerodynamic particle size such as the NEXT GENERATION IMPACTOR™ (NGI™, MSP Corporation), for which the respirable fraction is defined by similar aerodynamic size, for example <6.4 μm. In some embodiments, a laser diffraction apparatus is used to determine particle size, for example, the laser diffraction apparatus disclosed in U.S. Pat. No. 8,508,732, which disclosure is incorporated herein in its entirety for its relevant teachings related to laser diffraction, wherein the volumetric median geometric diameter (VMGD) of the particles is measured to assess performance of the inhalation system. For example, in various embodiments cartridge emptying of ≥80%, 85%, or 90% and a VMGD of the emitted particles of <12.5 μm, <7.0 μm, or <4.8 μm can indicate progressively better aerodynamic performance.

Respirable fraction on fill (RF/fill) represents the percentage (%) of powder in a dose that is emitted from an inhaler upon discharge of the powder content filled for use as the dose, and that is suitable for respiration, i.e., the percent of particles from the filled dose that are emitted with sizes suitable for pulmonary delivery, which is a measure of microparticle aerodynamic performance. As described herein, a RF/fill value of 40% or greater than 40% reflects acceptable aerodynamic performance characteristics. In certain embodiments disclosed herein, the respirable fraction on fill can be greater than 50%. In an exemplary embodiment, a respirable fraction on fill can be up to about 80%, wherein about 80% of the fill is emitted with particle sizes <5.8 μm as measured using standard techniques.

As used herein, the term “dry powder” refers to a fine particulate composition that is not suspended or dissolved in a propellant, or other liquid. It is not meant to necessarily imply a complete absence of all water molecules.

As used herein, “amorphous powder” refers to dry powders lacking a definite repeating form, shape, or structure, including all non-crystalline powders.

The present disclosure also provides improved powders comprising microcrystalline particles, compositions, methods of making the particles, and therapeutic methods that allow for improved delivery of drugs to the lungs for treating diseases and disorders in a subject and decreases the adverse effects caused by enteral or intravenous therapy. Embodiments disclosed herein achieve improved delivery by providing crystalline diketopiperazine compositions comprising microcrystalline diketopiperazine particles having high capacity for drug adsorption yielding powders having high drug content of one or more active agents. Powders made with the present microcrystalline particles can deliver increased drug content in lesser amounts of powder dose, which can facilitate drug delivery to a patient. The powders can be made by various methods, including methods utilizing surfactant-free solutions or solutions comprising surfactants depending on the starting materials.

In alternate embodiments disclosed herein, the drug delivery system can comprise a dry powder for inhalation comprising a plurality of substantially uniform, microcrystalline particles, wherein the microcrystalline particles can have a substantially hollow spherical structure and comprise a shell which can be porous comprising crystallites of a diketopiperazine that do not self-assemble in a suspension or in solution. In certain embodiments, the microcrystalline particles can be substantially hollow spherical and substantially solid particles comprising crystallites of the diketopiperazine depending on the drug and/or drug content provided and other factors in the process of making the powders. In one embodiment, the microcrystalline particles comprise particles that are relatively porous, having average pore volumes of about 0.43 cm³/g, ranging from about 0.4 cm³/g to about 0.45 cm³/g, and average pore size ranging from about 23 nm to about 30 nm, or from about 23.8 nm to 26.2 nm as determined by BJH adsorption.

Certain embodiments disclosed herein comprise dry powders comprising, a plurality of substantially uniform, microcrystalline particles, wherein the particles have a substantially spherical structure comprising a shell which can be porous, and the particles comprise crystallites of a diketopiperazine that do not self-assemble in suspension or solution and have a volumetric median geometric diameter less than 5 μm; or less than 2.5 μm and comprise an active agent.

In another embodiment herein, up to about 92% of the microcrystalline particles have a volumetric median geometric diameter of 5.8 μm. In one embodiment, the particle's shell is constructed from interlocking diketopiperazine microcrystals having one or more drugs adsorbed on their surfaces. In some embodiments, the particles can entrap the drug in their interior void volume and/or combinations of the drug adsorbed to the crystallites' surface and drug entrapped in the interior void volume of the spheres.

In certain embodiments, a diketopiperazine composition comprising a plurality of substantially uniformly formed, microcrystalline particles is provided, wherein the particles have a substantially hollow spherical structure and comprise a shell comprising crystallites of a diketopiperazine that do not self-assemble; wherein the particles are formed by a method comprising the step of combining diketopiperazine having a trans isomer content ranging from about 45% to 65% in a solution and a solution of acetic acid without the presence of a surfactant and concurrently homogenizing in a high shear mixer at high pressures of up to 2,000 psi to form a precipitate; washing the precipitate in suspension with deionized water; concentrating the suspension and drying the suspension in a spray drying apparatus. The microcrystalline particles can be pre-formed without for later used or combined with an active agent in suspension prior to spray drying.

The method can further comprise the steps of, adding with mixing a solution comprising an active agent or an active ingredient such as a drug or bioactive agent along with other pharmaceutically acceptable carriers and/or excipients prior to drying the solution or suspension, for example, prior to the spray drying step. In this manner, the active agent or active ingredient is adsorbed and/or entrapped on or within the particles. Particles made by this process can be in the submicron size range prior to spray-drying.

In certain embodiments, a diketopiperazine composition comprising a plurality of substantially uniformly formed, microcrystalline particles is provided, wherein the particles have a substantially hollow spherical structure and comprise a shell comprising crystallites of a diketopiperazine that do not self-assemble, and the particles have a volumetric mean geometric diameter less than or equal to 5 μm; wherein the particles are formed by a method comprising the step of combining diketopiperazine in a solution and a solution of acetic acid without the presence of a surfactant and concurrently homogenizing in a high shear mixer at high pressures of up to 2,000 psi to form a precipitate; washing the precipitate in suspension with deionized water; concentrating the suspension and drying the suspension in a spray drying apparatus.

The method can further comprise the steps of adding with mixing a solution comprising an active agent or an active ingredient such as a drug or bioactive agent prior to the spray drying step so that the active agent or active ingredient is adsorbed and/or entrapped on or within the particles. Particles made by this process can be in the submicron size range prior to spray-drying.

In certain embodiments, a diketopiperazine composition comprising a plurality of substantially uniformly formed, microcrystalline particles is provided, wherein the microcrystalline particles have a substantially hollow spherical structure and comprise a shell comprising crystallites of a diketopiperazine that do not self-assemble, and the particles have a volumetric mean geometric diameter less than equal to 5 μm; wherein the particles are formed by a method comprising the step of combining diketopiperazine in a solution and a solution of acetic acid without the presence of a surfactant and without the presence of an active agent, and concurrently homogenizing in a high shear mixer at high pressures of up to 2,000 psi to form a precipitate; washing the precipitate in suspension with deionized water; concentrating the suspension and drying the suspension in a spray drying apparatus.

In certain embodiments wherein the starting material comprising the active ingredient is an extract exhibiting a high degree of viscosity, or a substance having a honey like viscous appearance, the microcrystalline particles are formed as above and by washing them in water using tangential flow filtration (TFF) prior to combining with the extract or viscous material. After washing in water, the resultant particle suspension is lyophilized to remove the water and re-suspended in an alcohol solution, including ethanol or methanol prior to adding the active ingredient as a solid, or in a suspension, or in solution. In one embodiment, optionally, the method of making the composition comprises the step of adding any additional excipient, including one or more, amino acid, such as leucine, isoleucine, norleucine, methionine or one or more phospholipids, for example, 1,2-dipalmitoyl-sn-glycero-3-phosphocholine (DPPC) or 1,2-distearoyl-sn-glycero-3-phosphocholine (DSPC), concurrently with the active ingredient or subsequent to adding the active ingredient, and prior to spray drying. In certain embodiments, forming the composition comprises the step wherein the extract comprising desired active agents is, optionally, filtered or winterized to separate and remove layers of unwanted materials such as lipids to increase its solubility.

The method can further comprise the steps of adding a solution with mixing to the mixture, and wherein the mixing can optionally be performed with or without homogenization in a high shear mixer, wherein the solution comprises an active agent or an active ingredient such as a drug or bioactive agent prior to the spray drying step so that the active agent or active ingredient is adsorbed and/or entrapped within or on the surface of the particles. Particles made by this process can be in the submicron size range prior to spray-drying, or the particles can be formed from the solution during spray-drying.

In some embodiments herewith, the drug content can be delivered on crystalline powders using FDKP and which are lyophilized or sprayed dried at contents to about 10%, or about 20%, or about 30% or higher. In embodiments using microcrystalline particles formed from FDKP, or FDKP disodium salt, and wherein the particles do not self-assemble and comprise submicron size particles, drug content can typically be greater than 0.01% (w/w). In one embodiment, the drug content to be delivered with the microcrystalline particles is from about 0.01% (w/w) to about 75% (w/w); from about 1% to about 50% (w/w), from about 10% (w/w) to about 25% (w/w), or from about 10% to about 20% (w/w), or from 5% to about 30%, or greater than 25% depending on the drug to be delivered. An example embodiment wherein the drug is a nintedanib, the percent Compound I, nintedanib or pirfenidone in the composition can comprise from about 1% to about 50% (w/w) of the dry powder content. In certain embodiments, the drug content can be greater in the dry powder composition and can vary depending on the form and size of the drug particles to be delivered.

In an exemplary embodiment, a method of treating interstitial lung disease comprises a dry powder composition comprising microcrystalline particles of fumaryl diketopiperazine, wherein the Compound I, nintedanib, pirfenidone, or treprostinil is adsorbed to the particles and wherein the content of the Compound I, nintedanib, pirfenidone, or treprostinil in the composition comprises up to about 20% (w/w), or about 30% (w/w) and ranges from about 0.5% (w/w) to about 20% (w/w) or from about 1% (w/w) to about 10% (w/w), or from about 1% (w/w) to about 5% (w/w) of the dry powder.

In one embodiment, the composition herein can comprise one or more than one excipient suitable for inhalation, including, amino acids, including methionine, histidine, isoleucine and leucine. In this embodiment, for example, a treprostinil, nintedanib, pirfenidone, or Compound I composition can be used in the prevention and treatment of pulmonary fibrosis or pulmonary hypertension and interstitial lung disease by having the patient self-administer an effective dose comprising about 1 mg to 15 mg of a dry powder composition comprising microcrystalline particles of fumaryl diketopiperazine and treprostinil in a single inhalation. In another embodiment, the treprostinil content in the formulation can be from about 1 μg to about 200 μg. In one embodiment, the dry powder content of the cartridges comprising treprostinil can be between 20 μg and 500 μg, such as 20 μg, 30 μg, 60 μg, 90 μg, 120 μg, 150 μg, 180 μg, 200 μg, 300 μg, or 500 μg per dose regimen.

In alternate embodiments, the pharmaceutically acceptable carrier for making dry powders can comprise any carriers or excipients useful for making dry powders and which are suitable for pulmonary delivery. Example of pharmaceutically suitable carriers and excipients include, sugars, including saccharides and polysaccharides, such as lactose, mannose, sucrose, mannitol, trehalose; citrates, amino acids such as glycine, L-leucine, isoleucine, trileucine, tartrates, methionine, vitamin A, vitamin E, zinc citrate, sodium citrate, trisodium citrate, sodium tartrate, sodium chloride, zinc chloride, zinc tartrate, polyvinylpyrrolidone, polysorbate 80, phospholipids, including, diphosphatidylcholine and the like. In some embodiments, the suitable carrier or excipient is polysorbate 80.

In one embodiment, a method of self-administering a dry powder formulation to one's lung(s) with a dry powder inhalation system is also provided. The method comprises: obtaining a dry powder inhaler in a closed position and having a mouthpiece; obtaining a cartridge comprising a pre-metered dose of a dry powder formulation in a containment configuration, wherein the dry powder comprises Compound I, nintedanib, or pirfenidone, or treprostinil; opening the dry powder inhaler to install the cartridge or capsule; closing the inhaler to effectuate movement of the cartridge to a dose position; placing the mouthpiece in one's mouth, and inhaling once deeply to deliver the dry powder formulation to the lungs in less than 6 seconds.

In another embodiment, a method of treating interstitial lung disease including idiopathic pulmonary fibrosis is disclosed with diketopiperazine-based microparticles as carriers or excipients. The method comprises the administration of an inhalable dry powder composition or formulation comprising, for example, a diketopiperazine having the formula 2,5-diketo-3,6-di(4-X-aminobutyl) piperazine, wherein X is selected from the group consisting of succinyl, glutaryl, maleyl, and fumaryl. In this embodiment, the dry powder composition can comprise a diketopiperazine salt for making amorphous powders. In still yet another embodiment, there is provided a dry powder composition or formulation, wherein the diketopiperazine is 2,5-diketo-3,6-di-(4-fumaryl-aminobutyl) piperazine with or without a pharmaceutically acceptable carrier, or excipient and the active agent.

An inhalation system for delivering a dry powder formulation to a patient's lung(s) is provided, the system comprises a high resistance dry powder inhaler configured to have flow conduits with a total resistance to flow in a dosing configuration ranging in value from 0.05 to about 0.200 (√kPa)/liter per minute. The dry powder inhaler can be provided comprising a dry powder formulation for single use that can be discarded after use, or with individual doses that are replaceable in a multiple use inhaler and the individual dose enclosure or containers can be discarded after use. Individual dose cartridges comprising the dry powder formulations can be provided in individual packages or multiple cartridge doses can be provided in blister packages.

In one embodiment, a dry powder inhalation kit is provided comprising, a dry powder inhaler as described above, one or more medicament cartridges comprising a dry powder formulation for treating a disorder or disease such as respiratory tract and lung disease, including pulmonary fibrosis, pulmonary arterial hypertension, cystic fibrosis, respiratory infections, cancer, and other systemic diseases, including, endocrine disease, including, diabetes and obesity.

Methods of treating a disease or disorder in a patient with the dry powder inhaler embodiments disclosed herewith is also provided. The method of treatment comprises providing to a patient in need of treatment a dry powder inhaler comprising a cartridge containing a dose of an inhalable formulation comprising an active ingredient selected from the group as described above and a pharmaceutical acceptable carrier and/or excipient; and having the patient inhale through the dry powder inhaler deeply for about 3 to 4 seconds or less than 6 seconds to deliver the dose to the patient's lung. In the method, the patient can resume normal breathing pattern thereafter. Treatment of interstitial lung disease can be sustained for a period of a week, two weeks, three weeks, and up to two months; wherein the administration of, for example, nintedanib to a patient can occur once or twice daily with up to 300 mg, with patient monitoring for any adverse side effects.

Dry powder pharmaceutical compositions provided herewith comprise kinase inhibitor molecules that target cellular proteins, including, platelet derived growth factor receptor (PDGFR), fibroblast growth factor receptor (FGFR), vascular endothelial growth factor receptor (VEGFR), colony stimulating factor-1 receptor (CSFIR), leucocyte-specific protein tyrosine kinase (Lck2), transforming growth factor beta (TGF-β) kinases, including, activin receptors, for example, TGF-β type I receptor kinases for activin, ALK-4, ALK-5 and ALK-7; epidermal growth factor receptor (EGFR) and derivatives thereof, analogs thereof; and/or combinations thereof are used in the dry powder formulation which are delivered using an inhaler or a nebulizer using a liquid diluent.

In alternate embodiments, the kinase inhibitor can be of any type, for example, a type I inhibitor can be, but not limited to ibrutinib, bosutinib, crizotinib, dasatinib, erlotinib, gefitinib, lapatinib, pazopanib, ruxolitinib, sunitinib, and vemurafenib. In some embodiments, the kinase inhibitor can be a type II inhibitor, including, imatinib, sorafenib, axitinib, and nilotinib. In other embodiments, the kinase inhibitor is a type III inhibitor, for example, trametinib and GnF2. In alternative embodiments, the kinase inhibitor is a type IV of type V inhibitor, for example, afatinib, ibrutinib and HK1-272.

In some embodiments, the dry powder pharmaceutical composition for inhalation and treating lung disease, including, fibrotic lung disease comprises one or more of the kinase inhibitors and can also and optionally be formulated to comprise one or more pharmaceutical acceptable carriers or excipients, including, a diketopiperazine.

In certain embodiments, the pharmaceutical composition can further comprise any molecule or compound which is suitable for treating idiopathic lung disease and can be present in the composition either alone, or in combination with other active agents. Examples of active agents, include but is not limited to, deoxyribonuclease I (DNase I) and granulocyte macrophage colony stimulating factors (GM-CSF), anti-inflammatories, including, kinase inhibitors such as tyrosine kinase inhibitor molecules and activin receptor-like kinase inhibitors. In embodiments herewith, the pharmaceutical formulation comprises, optionally, one or more pharmaceutically acceptable excipients and/or carriers. In this and other embodiments the pharmaceutical composition is provided to the patient in a container, capsule or cartridge for inhalation using a dry powder inhaler.

In one embodiment, an inhalable pharmaceutical formulation is disclosed comprising a dry powder comprising a pharmaceutically acceptable excipient, including, a diketopiperazine having the ability for form particles and a therapeutically effective dose of a compound that inhibits enzymatic activity of kinase associated receptor protein molecules, such as tyrosine kinase, thereby inhibiting phosphorylation of intracellular proteins needed in the signaling pathway resulting in activation of scar formation. The inhalable pharmaceutical formulation can optionally comprise, one or more pharmaceutically acceptable carriers and/or excipients. In this and other embodiments, the inhalable pharmaceutical formulation can be formulated to comprise a dose of one or more active agents in the formulation for delivering with an inhaler in an amount of up to 30 mg, for example, 1 mg, 2 mg, 3 mg, 4 mg, 5 mg, 6 mg, 7 mg, 8 mg, 9 mg, 10 mg, 12 mg, 15 mg, 20 mg, 25 mg of an inhalable dry powder per cartridge or capsule, and comprising, optionally, one or more pharmaceutically acceptable salt thereof, including, serine-kinase inhibitor, a tyrosine kinase inhibitor, including, Bruton's tyrosine kinase (BTK) inhibitor, inositol tyrosine kinase (ITK) inhibitor, Aurora kinase inhibitor, CDK kinase inhibitor, MAPP kinase inhibitor, activin receptor-like kinase inhibitor, a pharmaceutically acceptable carrier, and/or excipients thereof. Multiple cartridges can be administered per dosing session depending on the patient's need, and up to 300 mg of the active agent per day, which can be administered once or more than once times per day. In some embodiments and depending on the patient's needs, the dosing can further be administered twice, thrice or more times daily.

In a certain embodiment, a method is provided for treating disease of the lungs, including interstitial lung disease, for example, idiopathic pulmonary fibrosis, comprising, administering to a subject in need of treatment an inhalable composition comprising a kinase inhibitor molecule, including, a tyrosine kinase inhibitor, and a diketopiperazine of the formula:

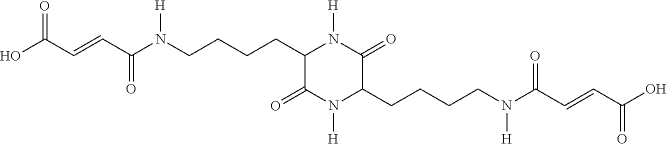 [SMILES: O=C(O)/C=C/C(=O)NCCCCC1NC(=O)C(CCCCNC(=O)/C=C/C(=O)O)NC1=O]

and optionally, one or more pharmaceutical excipients or carriers as defined above with respect to the formulation. In one embodiment, the kinase inhibitor molecule, includes, but not limited to axitinib, afatinib, bosutinib, cabozantinib, crizotinib, ceritinib, alectinib, dasatinib, brigatinib, ibrutinib, bosutinib, dastinib, nilotinib, ponatinib, cabozantinib, erlotinib, gefitinib, imatinib, lapatinib, nilotinib, pazopanib, regorafenib, ruxolitinib, sorafenib, sunitinib, vandetanib, vemurafenib, sirolimus, fasudil, riboviclib, idelalisib, midostaurin, piceatannol, trametinib, ruxoltinib, lenvatinib, regorafenib, tofacitinib, osimertinib, erdafitinib, and the like.

In another embodiment, the kinase inhibitor molecule is of the formula:

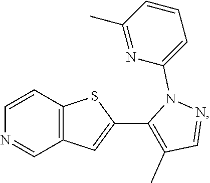 [SMILES: Cc1cccc(-n2ncc(C)c2-c2cc3cnccc3s2)n1]

a pharmaceutically acceptable salt, analog, or derivative, thereof, which molecule inhibits kinase activity associated with TGF-β receptor.

In one embodiment, a dry powder for inhalation is provided comprising crystalline particles of a diketopiperazine comprising a ALK-5 kinase inhibitor having the formula 2-[4-methyl-1-(6-methylpyridin-2-yl)-1H-pyrazol-5-yl]thieno-[3,2-c]pyridine, and/or a pharmaceutically acceptable salt, analog, or derivative thereof and one or more pharmaceutically acceptable excipients.

In one embodiment, a method of treating interstitial lung disease, including, IPF and/or scleroderma comprises administering to a patient in need of treatment a therapeutically effective amount of a composition comprising an inhalable composition comprising nintedanib in a dosage rage of from about 1 mg to about 25 mg per day by oral inhalation using a dry powder inhaler. In some embodiment, the treatment regimen is administered in single dose of one or more containers, capsules or cartridges comprising 1 mg, 2 mg, 3 mg, 4 mg, 5 mg, 6 mg, 7 mg, 8 mg, 10 mg, 12 mg, 14 mg, 16 mg, 18 mg, 20 mg, 24 mg, 30 mg, 35 mg, 40 mg, 50 mg, or more of nintedanib per inhalation as needed by the patient. In one aspect of this embodiment, the patient is treated in one or more sessions per day and preferably once, twice, or thrice daily for a period of a month or longer, wherein thereafter, the patient is evaluated for the effectiveness of treatment. In one embodiment herewith, the treatment is provided to the patient alone, as a single therapy, or in combination with alternative therapies, including, orally administered tablets or capsules of pirfenidone as recommended, or with reduced amounts or minimal dosage of non-inhalable/oral nintedanib, salt, ester or derivative thereof, or pirfenidone.

Methods for treating lung disease, including pulmonary fibrotic lung disease, fibrosing interstitial lung diseases (ILD) with a progressive phenotype, and systemic sclerosis-associated interstitial lung disease (SSc-ILD) comprises administering to a patient in need of treatment a therapeutically effective dose of a pharmaceutical composition comprising Compound I, Compound II/nintedanib, pirfenidone, a salt, ester, derivative thereof or analog thereof, or combination of one or more of the compounds for a period of a month or longer and determining the effectiveness of treatment by testing whether the patient pulmonary function is increased. Allowing the patient a refractory period and restarting the therapy as described above, wherein the refractory period can be a week, two weeks or up to a month or continuing to administer the therapy at reduced dosing amounts or as needed.

In one embodiment, a method of treatment comprises administering a therapeutically effective amount or dose of an inhalable pharmaceutical composition comprising a kinase such as Bruton's tyrosine kinase (BTK) inhibitor, including, Ibrutinib, a salt thereof, or a derivative thereof, and a pharmaceutically acceptable carrier or excipient, for example a diketopiperazine, such as those described above.

In some embodiments, a method of treatment comprises: administering a therapeutically effective amount or dose of an inhalable pharmaceutical composition comprising: a dry powder comprising a kinase inhibitor such as nintedanib, a salt thereof, or a derivative thereof; a pharmaceutically acceptable carrier or excipient such as a diketopiperazine, such as those described above; and polysorbate 80. In some embodiments, polysorbate 80 is considered a surfactant. In other embodiments, polysorbate 80 is considered an excipient or a carrier.

In some embodiments, the safety and tolerability of single and multiple-ascending doses of nintedanib inhalation powder (NIP) versus placebo in healthy adult participants was examined. In some embodiments, the safety and tolerability and efficacy of single and multiple-ascending doses of nintedanib inhalation powder (NIP) versus placebo in adult participants having idiopathic pulmonary fibrosis is examined.

In some embodiments, no adverse effects were reported. In other embodiments, adverse events were reported. In some embodiments, adverse events include cough, dyspnea, bronchospasm, dysgeusia, reductions in FEV₁≥15% from baseline, abnormal clinically significant vital signs (heart rate, blood pressure, respiratory rate, oxygen saturation rate, and body temperature), changes in baseline in liver enzymes and bilirubin, changes from baseline in coagulation parameters. In some embodiments, only cough and/or reductions in FEV₁≥15% from baseline were reported. In some of these embodiments, pharmacokinetic (PK) parameters were calculated based on plasma concentration of nintedanib from the plasma concentration time as appropriate, and the PK parameters include, but are not limited to, maximum observed plasma drug concentration (Cₘₐₓ), time to reach Cₘₐₓ (Tₘₐₓ), minimum observed plasma drug concentration over a dosing interval (Cₘᵢₙ), observed plasma drug concentration at 12 hours after dose administration (C₁₂), time to reach Cₘᵢₙ (Tₘᵢₙ), average concentration (C_{avg}), last quantifiable concentration (Cₗₐₛₜ), time of Cₗₐₛₜ (Tₗₐₛₜ), area under the plasma concentration-time curve from time 0 to 12 hours (AUC₀₋₁₂), area under the plasma concentration-time curve from time 0 to the last measurable plasma concentration (AUC₀₋₁), area under the plasma concentration-time curve from time 0 to infinity (AUC_{0-∞}), area under the plasma concentration-time curve over a dosing interval (AUCₜₐᵤ), AUC extrapolated from the last plasma drug concentration to infinity in percent of the total AUC (fₑₓₜ), apparent total plasma clearance after extravascular administration (CL/F), apparent volume of distribution based on terminal phase after extravascular administration (V_{z}/F), apparent first-order terminal elimination rate constant (kₑₗ), terminal elimination phase half-life (t_{1/2}).

In some embodiments, nintedanib plasma concentrations increased with increasing dose after single dose administration of NIP.

In some embodiments, nintedanib was rapidly absorbed with maximum concentrations observed at the first measured sample immediately (within a few minutes) after the inhaled dose administration.

In some embodiments, terminal elimination half-life was similar between dose groups after once daily single-dose administration with geometric mean values ranging between 10.3 hours and 14.5 hours. In some embodiments, the calculated half-life values for a twice daily dosing regimen were generally shorter than those for a once daily single-dose administration, most likely due to the differing time range of data for calculation (24 hours after dose administration for once daily dosing and 12 hours after dose administration for twice daily dosing) and the multi-phasic nature of the plasma concentration versus time profiles.

In some embodiments, with NIP administered as a single-dose once daily, Cₘₐₓ concentrations increased as dose increased. In some embodiments, the geometric mean (% CV) Cₘₐₓ values was 4.10 ng/mL for a 2-mg dose group. In some embodiments, the geometric mean (% CV) Cₘₐₓ values was 6.84 ng/ml for a 4 mg dose group. In some embodiments, the geometric mean (% CV) Cₘₐₓ values was 27.2 ng/mL for an 8-mg dose group. In some embodiments, with NIP administered as a once daily single-dose, the geometric mean (% CV) Cₘₐₓ values was about 2 ng/ml to about 50 ng/ml, about 2-30 ng/mL, about 2-4 ng/mL, about 4-6 ng/mL, about 6-8 ng/ml, about 8-10 ng/ml, about 10-15 ng/mL, about 15-20 ng/mL, about 20-25 ng/mL, about 25-30 ng/ml, about 30-40 ng/mL, about 40-50 ng/mL, or any geometric mean (% CV) Cₘₐₓ value in a range bounded by any of these values.

In some embodiments, with NIP administered twice daily, Cₘₐₓ increased for a 2-mg dose but not a 4-mg dose: geometric mean (% CV) Cₘₐₓ values on Day 1 of 2.15 ng/ml (128.9%) for a 2-mg dose group and 17.9 ng/ml (28.9%) for a 4-mg dose group with values on Day 7 of 8.43 ng/ml (24.4%) for a 2-mg dose group and 11.4 ng/ml (42.7%) for a 4-mg dose group. In some embodiments, with NIP administered as twice daily, the geometric mean (% CV) Cₘₐₓ values was about 2 ng/ml to about 100 ng/ml, about 2-50 ng/ml, about 2-20 ng/mL, about 2-4 ng/ml, about 4-6 ng/ml, about 6-8 ng/ml, about 8-10 ng/mL, 8-12 ng/ml, about 5-15 ng/ml about 10-15 ng/ml, about 15-20 ng/ml, about 20-25 ng/mL, about 25-30 ng/ml, about 30-35 ng/mL, about 35-40 ng/ml, about 20-45 ng/ml about 40-45 ng/ml, about 45-50 ng/ml, about 50-60 ng/mL, about 60-70 ng/mL, about 70-80 ng/ml, about 80-90 ng/mL, about 90-100 ng/ml, or any geometric mean (% CV) Cₘₐₓ value in a range bounded by any of these values.

In some embodiments, with NIP administered twice daily, on Day 7, the calculated geometric mean AUCₜₐᵤ was 8.38 h×ng/ml for a 2-mg dose group and 14.9 h×ng/ml for a 4-mg dose group. In some embodiments, with NIP administered twice daily, the calculated geometric mean AUCₜₐᵤ was about 4 h×ng/ml to about 30 h×ng/mL, about 4-6 h×ng/ml, about 6-8 h×ng/ml, about 8-10 h×ng/ml, about 10-12 h×ng/mL, about 12-14 h×ng/ml, about 14-16 h×ng/ml, about 16-18 h×ng/ml, about 18-20 h×ng/ml, about 20-22 h×ng/ml, about 22-24 h×ng/ml, about 24-26 h×ng/ml, about 26-28 h×ng/mL, about 28-30 h×ng/ml, or any geometric mean AUCₜₐᵤ value in a range bounded by any of these values.

In some embodiments, with NIP administered twice daily, on Day 7 the geometric mean CLₛₛ/F was 239 L/h for a 2-mg dose group and 269 L/h for a 4-mg dose group. In some embodiments, with NIP administered twice daily, the geometric mean CLₛₛ/F was about 100 L/h to about 400 L/h, about 100-200 L/h, about 200-300 L/h, about 300-400 L/h, about 100-125 L/h, about 125-150 L/h, about 150-175 L/h, about 175-200 L/h, about 200-225 L/h, about 225-250 L/h, about 250-275 L/h, about 275-300 L/h, about 300-325 L/h, about 325-350 L/h, about 350-375 L/h, about 375-400 L/h, or any geometric mean CLₛₛ/F value in a range bounded by any of these values.

In some embodiments, with NIP administered twice daily, on Day 7, geometric mean V/F was 5,580 L for a 2-mg dose group and 5,310 L for a 4-mg dose group. In some embodiments, with NIP administered twice daily, geometric mean V/F was about 4000 L to about 8000 L, about 5000-7000 L, about 5000-6000 L, about 5300-5600 L, about 5200-5300 L, about 5300-5400 L, about 5400-5500 L, about 5500-5600 L, about 5600-5700 L, or any geometric mean V_{z}/F value in a range bounded by any of these values.

In some embodiments, with NIP administered twice daily, the geometric mean accumulation AUCₜₐᵤ ratios on Day 7 were 4.93 for a 2-mg dose group and 1.45 for a 4-mg dose group. In some embodiments, with NIP administered twice daily, the geometric mean accumulation AUCₜₐᵤ ratio was about 0.5 to about 10, about 0.5-1, about 1-1.5, about 1.5-2, about 2-3, about 3-4, about 4-5, about 4.5 to about 5.5, about 5-6, about 6-7, about 7-8, about 8-9, about 9-10, or any geometric mean accumulation AUCₜₐᵤ ratio in a range bounded by any of these values.

In some embodiments, with NIP administered twice daily, the geometric mean Cₘₐₓ accumulation on Day 7 was 3.91 for a 2-mg dose group and 0.637 for a 4-mg dose group. In some embodiments, with NIP administered twice daily, the geometric mean Cₘₐₓ accumulation was about 0.3 to about 8, about 0.3-1, about 1-2, about 2-3, about 3-4, about 4-5, about 5-6, about 6-7, about 7-8, about 0.5-0.8, about 3.5-4.5, about 0.5-4, about 3-4, or any geometric mean Cₘₐₓ accumulation in a range bounded by any of these values.

In some embodiments, with NIP administered once or twice daily, all TEAEs reported were mild and no SAEs, severe TEAEs, TEAEs leading to discontinuation or dose adjustments, or deaths were reported. In some embodiments, NIP is safe and well tolerated in healthy adults. In some embodiments, all TEAEs reported with NIP administered once or twice daily were considered related to the NIP treatment.

In some embodiments, only 2 types of AEs were reported and both were respiratory-related: cough and FEV reduction of ≥15%. In these embodiments, patients did not have any associated signs or other symptoms. In some embodiments, no GI (e.g., diarrhea, nausea, vomiting, abdominal discomfort) or neurologic (e.g., headache, fatigue) AEs were reported with NIP administered once or twice daily.

In some embodiments, no TEAEs or SAEs related to clinically significant vital signs abnormalities were reported with NIP administered once or twice daily. In some embodiments, reported abnormalities in vital signs did not show treatment-related trends. with NIP administered once or twice daily.

In some embodiments, no clinically significant ECG abnormalities or TEAEs related to ECG abnormalities were reported.

In some embodiments, the incidence of ≥15% reduction in FEV₁ from baseline and pre-dose were observed within 30 minutes of NIP administration (Dose 1 only). In some embodiments, no treatment-related trends were observed.

In some embodiments, neither cough nor ≥15% reduction in FEV₁ worsened with increased or ongoing dosing. In some embodiments, cough was more common after the first cartridge intake regardless of the overall dose.

In some embodiments, no clinically significant abnormal laboratory results were reported. In some embodiments, no TEAEs related to changes from baseline in liver enzymes, bilirubin, or coagulation parameters were reported.

The following Embodiments are contemplated.

### EMBODIMENTS

Embodiment 1. An inhalable pharmaceutical composition, comprising:
a dry powder comprising diketopiperazine particles and a therapeutically effective dose of nintedanib or Compound I having the formula:

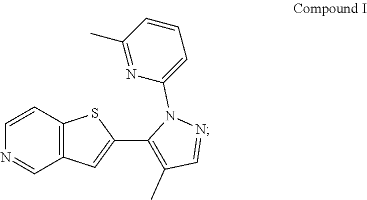 [SMILES: Cc1cccc(-n2ncc(C)c2-c2cc3cnccc3s2)n1]

or a pharmaceutically acceptable salt thereof and optionally, one or more pharmaceutically acceptable carriers and/or excipients.

Embodiment 2. The inhalable pharmaceutical composition of embodiment 1, wherein the therapeutically effective dose is in an amount of up to about 50 mg of nintedanib or Compound I and the one or more pharmaceutically acceptable carriers and/or excipients.

Embodiment 3. The inhalable pharmaceutical composition of embodiment 1, wherein the one or more pharmaceutically acceptable carrier and/or excipient is a surfactant, an amino acid, or a phospholipid.

Embodiment 4. The inhalable pharmaceutical composition of embodiment 1, wherein the diketopiperazine is of the formula:

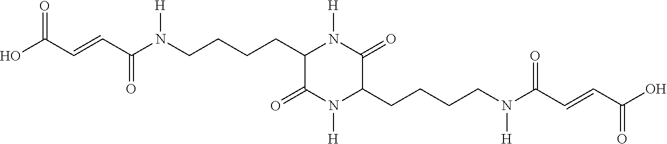 [SMILES: O=C(O)/C=C/C(=O)NCCCCC1NC(=O)C(CCCCNC(=O)/C=C/C(=O)O)NC1=O]

in crystalline, or microcrystalline particle form.

Embodiment 5. The inhalable pharmaceutical composition of embodiment 1, wherein the therapeutically effective dose of nintedanib or a pharmaceutically acceptable salt thereof, or Compound I or a pharmaceutically acceptable salt thereof, ranges from about 1 mg to about 50 mg in the dry powder composition.

Embodiment 6. The inhalable pharmaceutical composition of embodiment 1, wherein the dry powder composition is an amorphous powder.

Embodiment 7. The inhalable pharmaceutical composition of claim 1, wherein the dry powder comprises one or more pharmaceutically acceptable carriers and/or excipients is selected from lactose, mannose, sucrose, mannitol, trehalose, sodium citrate, trisodium citrate, zinc citrate, glycine, L-leucine, isoleucine, trileucine, sodium tartrate, zinc tartrate, methionine, vitamin A, vitamin E, sodium chloride, zinc chloride, polyvinylpyrrolidone and polysorbate 80.

Embodiment 8. The inhalable pharmaceutical composition of embodiment 7, wherein the dry powder comprises one or more pharmaceutically acceptable carriers and/or excipients selected from the group consisting of sodium citrate, sodium chloride, leucine or isoleucine and trehalose.

Embodiment 9. The inhalable pharmaceutical composition of embodiment 3, wherein the surfactant is polysorbate 80.

Embodiment 10. The inhalable pharmaceutical composition of embodiment 4, wherein microcrystalline particles have a specific surface area ranging from about 25 m²/g to about 63 m²/g.

Embodiment 11. The inhalable pharmaceutical dry powder composition of embodiment 4, wherein microcrystalline particles have a pore size ranging from about 23 nm to about 30 nm.

Embodiment 12. A method of treating idiopathic pulmonary fibrosis comprising:
administering to a patient in need of treatment by oral inhalation a dry powder composition comprising crystalline diketopiperazine particles and up to 50 mg of nintedanib or a pharmaceutically acceptable salt thereof, or Compound I of the formula:

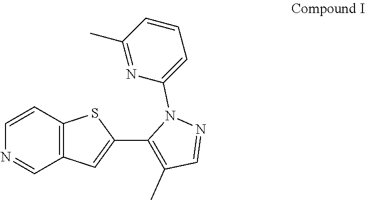 [SMILES: Cc1cccc(-n2ncc(C)c2-c2cc3cnccc3s2)n1]

or a pharmaceutically acceptable salt thereof;
and, optionally, one or more pharmaceutically acceptable carriers and/or excipients, wherein the dry powder composition is provided in a dry powder inhaler.

Embodiment 13. The method of embodiment 12, wherein a therapeutically effective dose of the dry powder composition is provided to said patient in one or more capsules or cartridges for adapting to said dry powder inhaler prior use and wherein each capsule or cartridge comprises up to 30 mg of nintedanib or Compound I.

Embodiment 14. The method of embodiment 13, wherein the therapeutically effective dose comprises up to 300 mg of nintedanib or Compound I per day provided in multiple cartridges.

Embodiment 15. The method of embodiment 12, wherein the one or more pharmaceutically acceptable carriers and/or excipients is selected from the group consisting of fumaryl diketopiperazine, lactose, mannose, sucrose, mannitol, trehalose, sodium citrate, trisodium citrate, zinc citrate, glycine, L-leucine, isoleucine, trileucine, sodium tartrate, zinc tartrate, methionine, vitamin A, vitamin E, sodium chloride, zinc chloride, polyvinylpyrrolidone, and polysorbate 80.

Embodiment 16. The method of embodiment 12, wherein the one or more pharmaceutically acceptable carriers and/or excipients is polysorbate 80.

Embodiment 17. The method of embodiment 12, wherein the diketopiperazine is fumaryl diketopiperazine.

Embodiment 18. The method of embodiment 12, wherein the dry powder composition is administered in at least one inhalation in less than 10 seconds per cartridge.

Embodiment 19. The method of embodiment 12, comprising the inhalable pharmaceutical composition of embodiment 1, wherein the dry powder composition comprises about 2 mg, about 4 mg, about 6 mg, about 8 mg, or about 10 mg of nintedanib or a pharmaceutically acceptable salt thereof.

Embodiment 20. A dry powder inhaler comprising a movable member for mounting a cartridge and configuring a container to attain a dosing configuration, wherein said cartridge comprises the dry powder composition of embodiment 1.

The following examples illustrate some of the processes for making dry powders suitable for using with the inhalers described herein and data obtained from experiments using the dry powders.

### Example 1

### Preparation of Crystalline Composite Nintedanib Dry Powders

A 10% nintedanib solution (concentration of nintedanib in this solution could range from 1% nintedanib to 35% (w/w) nintedanib) was prepared by adding nintedanib (0.025 g) to a 10% (w/w) acetic acid solution (0.225 g) (concentration of acetic acid solution could range from 10% to 100% acetic acid). The nintedanib solution was added to a microcrystalline particle (XC) suspension (1.31% solids, 188.93 g) suspension of 3,6-bis(N-fumaryl-4-aminobutyl)-2,5-diketopiperazine, or fumaryl diketopiperazine (solids content of the XC suspension could range from 0.5% to 5% (w/w)). The nintedanib XC suspension was spray dried using a Buchi B-290 spray dryer with the conditions shown in Table 1 to produce a 1% (w/w) nintedanib XC powder and yield was about 2.5 g.

TABLE 1
Nintedanib Powder Spray Drying Conditions

| Spray Dryer Parameter | Set Point | |
|---|---|---|
| Inlet Temperature | 180° | C. |
| Aspirator Pump Speed | 90% | |
| Feed Pump Speed | 25% | |
| Nitrogen Flow | 60 | mm |

Preparation 20% (w/w) Nintedanib Crystalline XC Powder Preparations

A 10% nintedanib solution (concentration of nintedanib in this solution could range from 1% nintedanib to 35% (w/w) nintedanib) was prepared by adding nintedanib (3.33 g) to a 20% acetic acid solution (29.97 g) (concentration of acetic acid solution could range from 10% to 100% acetic acid). Separately, an XC suspension was prepared by adding fumaryl diketopiperazine particles (11.67 g) to deionized water (705.03 g) (suspension solids=1.63%) (solids content of the XC suspension could range from 0.5% to 5%). The nintedanib solution was then added to the XC suspension and the resulting nintedanib XC suspension was spray dried using a Buchi B-290 spray dryer with the conditions shown in Table 1 to produce a 20% nintedanib XC powder and a resultant yield of about 15 g.

### Preparation of Crystalline Nintedanib T Dry Powders

A 10% nintedanib solution (concentration of nintedanib in this solution could range from 1% nintedanib to 35% (w/w) nintedanib) was prepared, for example, by adding nintedanib (0.025 g) (nintedanib charge could range from 0.025 g to 50 g) to a 10% acetic acid solution (0.225 g) (concentration of acetic acid solution could range from 10% to 100% acetic acid). The nintedanib solution was added to a suspension of 3,6-bis(N-fumaryl-4-aminobutyl)-2,5-diketopiperazine pre-formed particles (T suspension; 8.11% solids, 30.52 g) (solids content of the T suspension could range from 0.5% to 20% (w/w)) as described below. The nintedanib T suspensions were then dried either by spray drying or by lyophilization to produce 1% nintedanib T powders. Spray dried powders were dried using a Buchi B-290 spray dryer with conditions shown in Table 1. Lyophilized powders were prepared by first pelletizing the nintedanib T suspension into liquid nitrogen followed by drying in a Virtis Genesis 25 XL shelf lyophilizer. The lyophilizer was run on a program where the shelf temperature was ramped from −45° C. to 25° C. at 0.2° C./min and then maintained at 25° C. under vacuum until the powder was completely dried and the resultant yield was about 2.5 g.

Preparation of Spray Dried 20% (w/w) Nintedanib Crystalline T Powder

A 10% nintedanib solution (concentration of nintedanib in this solution could range from 1% nintedanib to 35% nintedanib) was prepared by adding nintedanib (3.33 g) to a 20% (w/w) acetic acid solution (30.0 g) (concentration of acetic acid solution could range from 10% to 100% acetic acid). The nintedanib solution was added to a T suspension (8.99% solids, 129.81 g) (solids content of the T suspension could range from 0.5% to 20 wt %). The nintedanib T suspension was then spray dried using a Buchi B-290 spray dryer with conditions shown in Table 1. The resultant yield was about 15 g.

### Preparation of Lyophilized 20% Nintedanib T Powder

A 10% nintedanib solution (concentration of nintedanib in this solution could range from 1% nintedanib to 35% (w/w) nintedanib) was prepared by adding nintedanib (2.63 g) to a 10% acetic acid solution (23.63 g) (concentration of acetic acid solution could range from 10% to 100% acetic acid). The nintedanib solution was added to a T suspension (8.99% solids, 104.23 g) (solids content of the T suspension could range from 0.5% to 20 wt %. The nintedanib T suspension was lyophilized by first pelletizing the nintedanib T suspension into liquid nitrogen followed by drying in a Virtis Genesis 25 XL shelf lyophilizer. The lyophilizer was run on a program where the shelf temperature was ramped from −45° C. to 25° C. at 0.2° C./min and then maintained at 25° C. under vacuum until the powder was completely dried and resulted in about 12 g yield.

Preparation of Lyophilized 20% Nintedanib T Powder with Reduced Solids Content

A 10% nintedanib solution (concentration of nintedanib in this solution could range from 1% nintedanib to 35% (w/w) nintedanib) was prepared by adding nintedanib (3.09 g*) to a 20% acetic acid solution (27.81 g) (concentration of acetic acid solution could range from 10% to 100% acetic acid). Separately, the T suspension (8.99% solids, 132.48 g*) was diluted with deionized water (136.62 g) (solids content of the T suspension could range from 0.5% to 20% (w/w)). The nintedanib solution was added to this diluted T suspension resulting in a nintedanib T suspension with a solids content of 5.00%. The nintedanib T suspension was lyophilized by first pelletizing it into liquid nitrogen followed by drying in a Virtis Genesis 25 XL shelf lyophilizer. The lyophilizer was run on a program where the shelf temperature was ramped from −45° C. to 25° C. at 0.2° C./min and then maintained at 25° C. under vacuum until the powder was completely dried and the resultant yield was about 15 g.

Preparation of Lyophilized 20% Nintedanib T Powder with Reversed Component Addition

A 10% nintedanib solution (concentration of nintedanib in this solution could range from 1% nintedanib to 35% (w/w) nintedanib) was prepared by adding nintedanib (3.09 g) to a 20% acetic acid solution (27.81 g) (concentration of acetic acid solution could range from 10% to 100% acetic acid). The nintedanib solution was diluted with deionized water (97.19 g). Lyophilized T particles (11.91 g*) were then added to the nintedanib solution, portionwise, over 4 min. Deionized water (10.00 g) was used to wash the residual lyophilized T particles into the nintedanib T suspension. The nintedanib T suspension was lyophilized by first pelletizing it into liquid nitrogen followed by drying in a Virtis Genesis 25 XL shelf lyophilizer. The lyophilizer was run on a program where the shelf temperature was ramped from −45° C. to 25° C. at 0.2° C./min and then maintained at 25° C. under vacuum until the powder was completely dried and resultant yield was about 15 g.

### Preparation of Lyophilized 20% Nintedanib Esylate T Powder

A 1% nintedanib esylate solution (concentration of nintedanib esylate in this solution could range from 1% nintedanib esylate to 5% nintedanib esylate) was prepared by adding nintedanib esylate (3.61 g*) portionwise to deionized water (357.39 g). The nintedanib esylate solution was added to the T suspension (8.99% solids, 126.70 g*) (solids content of the T suspension could range from 0.5% to 20%) and the resulting nintedanib esylate T suspension was pelletized into liquid nitrogen followed by drying in a Virtis Genesis 25 XL shelf lyophilizer. The lyophilizer was run on a program where the shelf temperature was ramped from −45° C. to 25° C. at 0.2° C./min and then maintained at 25° C. under vacuum until the powder was completely dried and resultant yield was about 15 g.

Powder Testing—Powders were evaluated for geometric particle size distribution using a Sympatec laser diffraction instrument fitted with a RODOS bulk powder dispersing system. Bulk powders were dispersed at 0.5 bar and 3.0 bar. Powders were also evaluated for aerodynamic particle size distribution using an Andersen Cascade impactor (ACI). Powders were discharged through the ACI from Gen 2C cartridges (10 mg cartridge fills) at 4 kPa. Data for the nintedanib powders are shown in Table 2.

TABLE 2
Nintedanib Powder Data

| | | | | | RODOS DATA | | | | ACI Data | |
|---|---|---|---|---|---|---|---|---|---|---|
| | | % | | Ninte. | 0.5 bar | | 3.0 bar | | Avg. | |
| % Powder | Yield | Solid | Yield | Assay | x(50) | x(90) | x(50) | x(90) | CE | RF |
| content | (g) | (%) | (%) | (Wt %) | (μm) | (μm) | (μm) | (μm) | (%) | (%) |

XC Powders

| | | | | | | | | | | |
|---|---|---|---|---|---|---|---|---|---|---|
| 1% Nintedanib | 2.5 | 1.32 | 78.4 | 1.01 | 4.34 | 8.54 | 3.14 | 7.94 | 89.1 | 27.9 |
| 1% Nintedanib | 2.5 | 1.32 | 77.6 | 0.95 | 4.19 | 9.12 | 3.33 | 10.09 | 75.7 | 25.0 |
| 10% Nintedanib | 2.5 | 2.06 | 80.0 | 8.52 | 2.19 | 5.00 | 1.73 | 4.30 | 95.2 | 45.4 |
| 20% Nintedanib | 2.5 | 2.26 | 82.8 | 17.94 | 2.41 | 5.55 | 1.89 | 5.21 | 90.9 | 46.1 |
| 20% Nintedanib | 15.0 | 2.00 | 67.0 | 20.70 | 2.14 | 5.82 | 1.51 | 3.69 | 83.9 | 41.7 |

Spray Dried T Powders

| | | | | | | | | | | |
|---|---|---|---|---|---|---|---|---|---|---|
| 1% Nintedanib | 2.5 | 8.12 | 80.0 | 1.08 | 1.54 | 3.60 | 1.31 | 2.44 | 91.8 | 72.2 |
| 1% Nintedanib | 2.5 | 8.12 | 79.2 | 0.95 | 1.50 | 3.34 | 1.35 | 2.65 | 79.7 | 53.8 |
| 10% Nintedanib | 2.5 | 7.46 | 82.8 | 8.37 | 1.74 | 3.81 | 1.42 | 2.73 | 76.2 | 43.7 |
| 20% Nintedanib | 2.5 | 7.68 | 82.8 | 18.07 | 1.94 | 4.33 | 1.52 | 2.87 | 87.5 | 36.7 |
| 20% Nintedanib | 15 | 9.19 | 88.5 | 20.30 | 2.01 | 4.32 | 1.41 | 2.79 | 78.6 | 48.6 |

Lyophilized T Powders

| | | | | | | | | | | |
|---|---|---|---|---|---|---|---|---|---|---|
| 1% Nintedanib | 2.5 | 8.12 | 97.2 | 1.15 | 1.75 | 4.58 | 1.33 | 2.48 | 97.2 | 60.6 |
| 1% Nintedanib | 2.5 | 8.12 | 97.6 | 0.88 | 2.09 | 5.05 | 1.58 | 3.13 | 92.1 | 54.0 |
| 10% Nintedanib | 2.5 | 7.46 | 95.6 | 9.27 | 1.98 | 4.27 | 1.48 | 2.84 | 62 | 38.2 |
| 20% Nintedanib | 2.5 | 7.68 | 94.4 | 18.28 | 2.45 | 5.41 | 1.71 | 3.28 | 88 | 45.1 |
| 20% Nintedanib | 12 | 9.20 | 94.4 | 21.30 | 3.78 | 9.05 | 1.89 | 4.31 | 87.3 | 32.2 |
| 20% Nintedanib | 15 | 5.00 | 98.8 | 20.72 | 2.76 | 6.28 | 1.95 | 3.94 | 83.9 | 36.4 |
| 20% Nintedanib | 15 | 10.00 | 97.6 | 20.19 | 3.09 | 7.72 | 2.02 | 4.17 | 83.3 | 33.4 |
| Reverse Addition | | | | | | | | | | |
| 20% Nintedanib | 15 | 3.08 | 100.9 | 18.99 | 3.76 | 9.76 | 2.13 | 5.09 | 50.2 | 6.1 |
| Esylate | | | | | | | | | | |

Table 2 shows the target yield (Yield (g)) for the process. The percent yield (Yield (%) indicates the percent of the target yield recovered from the process. As can be seen in Table 2, the process product yield was greater than about 67% in the composition reactions for both sprayed dried or lyophilized dry powders. In addition, it can be seen that the percent yield was improved for the lyophiplized T powders, and all powders containing 10 wt % and 20 wt % nintedanib in the composition, no matter the method of making the powders. The data show the average powder delivered from the delivery system was greater than 75% for all the XC powder and spray-dried T powders, and greater than or equal to 62% for all lyophilized T powders, as assessed by cartridge emptying (CE) measurements with some powders yielding upwards of about 97% CE. The data also illustrates that the XC powders at higher concentration (10 wt % and 20 wt %) appear to have consistent cartridge emptying performance than at lower concentrations, however the best CE performance powders were the 1% T powders either sprayed dried or lyophilized.

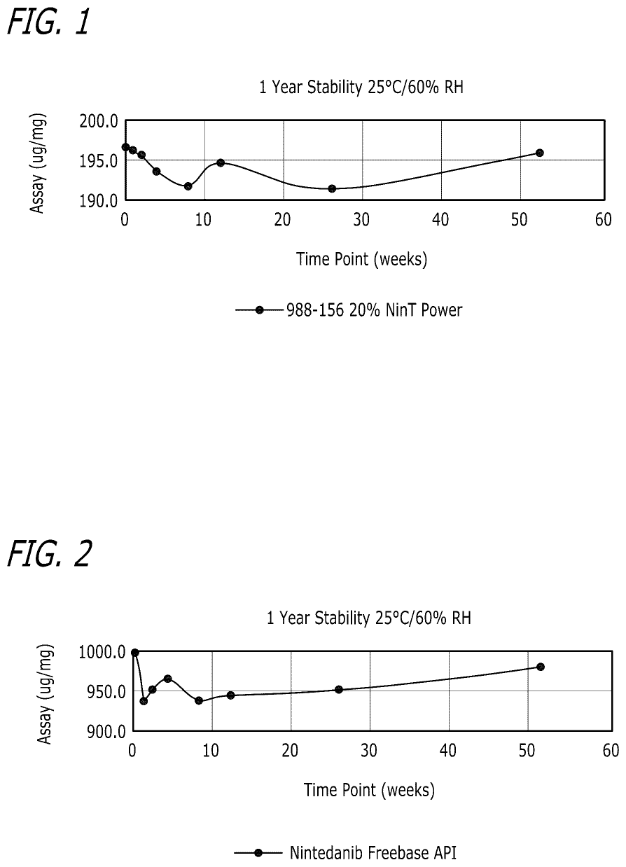

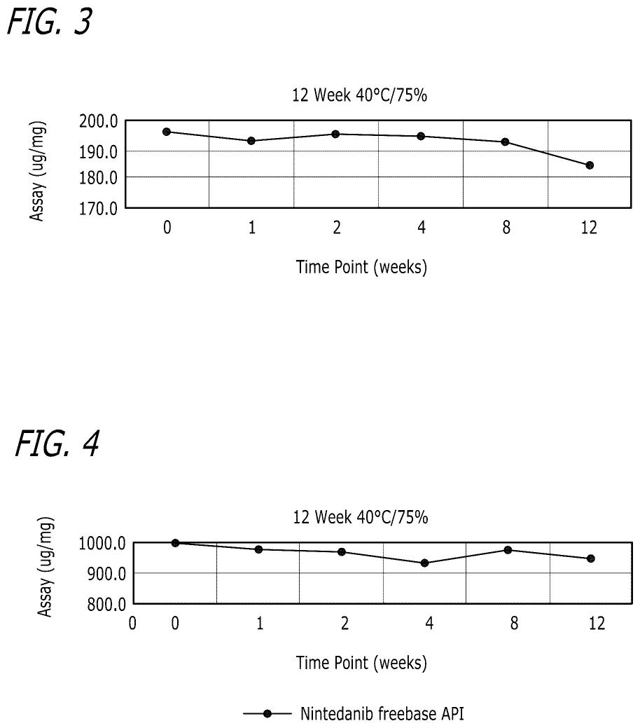

Sample powders were taken for room temperature stability at 25° C., 60% relative humidity (RH) testing for a year as well as incubated at 40° C., 75% RH for a period of 12 weeks. Test samples were taken at various times from the start of the experiment and assay of nintedanib content. Parallel samples were also carried out with nintedanib free base compound alone for comparison. Results from the experiments are illustrated in FIG. 1, FIG. 2, FIG. 3, and FIG. 4. FG. 1 and FIG. 2 depict the room temperature stability results of the experiments wherein the stability of the nintedanib formulated in the crystalline (T powder) as described above is similar to the nintedanib free base (FIG. 2) compound for the duration of the experiments, but with less variation in content in the powder formulated T powder (NinT). (FIG. 1). Similarly, the T powder formulated with nintedanib (FIG. 3) that were tested at extreme heat of 40° C., 75% RH for 12 weeks compared to the nintedanib free base compound (FIG. 4) appear to have similar stability profiles and very stable for long periods of times or with a loss of less than about 5% of the original content of nintedanib in the composition.

### Example 2

### Preparation of Crystalline Composite Pirfenidone Dry Powders

A 25% pirfenidone solution (concentration of pirfenidone in this solution can range from 1% pirfenidone to 40% pirfenidone) was prepared by adding pirfenidone (0.20 g) (the pirfenidone charge used to prepare these powders was varied between 0.2 g and 0.63 g) to ethanol (0.60 g) (when a 25% pirfenidone solution was used, water could be added to the ethanol up to a 50:50 weight ratio of ethanol:water). The pirfenidone solution was added to a microcrystalline (XC) suspension (1.31% solids, 137.40 g) (solids content of the XC suspension could range from 0.5% to 5%). The pirfenidone XC suspension was spray dried using a Buchi B-290 spray dryer with the conditions shown in Table 1 to produce a pirfenidone XC powder.

### Preparation of Crystalline Pirfenidone Powders

A 25% pirfenidone solution (concentration of pirfenidone in this solution could range from 1% pirfenidone to 40% pirfenidone) was prepared by adding pirfenidone (0.20 g) (the pirfenidone charge used to prepare these powders was varied between 0.2 g and 0.33 g) to ethanol (0.60 g) (when a 25% pirfenidone solution was used, water could be added to the ethanol up to a 50:50 weight ratio of ethanol:water). The pirfenidone solution was added to a T suspension (8.11% solids, 22.19 g) (solids content of the T suspension could range from 0.5% to 20%). The pirfenidone T suspensions were then dried either by spray drying or by lyophilization to produce pirfenidone T powders. Spray dried powders were dried using a Buchi B-290 spray dryer with conditions shown in Table 1. Lyophilized powders were prepared by first pelletizing the pirfenidone T suspension into liquid nitrogen followed by drying in a Virtis Genesis 25 XL shelf lyophilizer. The lyophilizer was run on a program where the shelf temperature was ramped from −45° C. to 25° C. at 0.2° C./min and maintained at 25° C. under vacuum until the powder was completely dried.

### Preparation of Amorphous Pirfenidone Powders

A 25% pirfenidone solution (concentration of pirfenidone in this solution could range from 1% pirfenidone to 40% pirfenidone) was prepared by adding pirfenidone (0.20 g) (the pirfenidone charge used to prepare these powders was varied between 0.2 g and 0.22 g) to ethanol (0.60 g) (when a 25% pirfenidone solution was used, water could be added to the ethanol up to a 50:50 weight ratio of ethanol:water). Separately a 10% FDKP-disodium salt solution was prepared. Leucine (leucine charge ranged from 0 g to 0.45 g), and FDKP-disodium salt (1.80 g) were dissolved in in deionized water (16.20 g) (concentration of the FDKP-disodium salt in this solution could range from 5% to 20%). The pirfenidone solution was added to the FDKP-disodium salt solution and the resulting solution was spray dried using a Buchi B-290 spray dryer run using the conditions shown in Table 1 to produce pirfenidone amorphous powders.

### Example 3

### Preparation of Crystalline Composite (XC) Dry Powders Using Compound I

15% (w/w) powder preparations for target yield of 2.5 g were prepared. A 15% of Compound I solution (concentration of Compound I in this solution could range from 1% to 15%) was prepared by adding Compound I (0.125 g) (Compound I charge could range from 0.025 g to 0.75 g) to a 50% acetic acid solution (0.708 g) (concentration of acetic acid solution could range from 50% to 100% acetic acid). The Compound I solution was added to an XC suspension (1.31% solids, 181.30 g) (solids content of the XC suspension could range from 0.5% to 5%) (Suspension charge would be adjusted to obtain the desired Compound I charge in the final powder). The Compound I XC suspension was spray dried using a Buchi B-290 spray dryer with the conditions shown in Table 3 to produce the desired Compound I XC powder.

20% (w/w) XC powder preparations to target yield of 20 g: Powders were prepared for a target yield of 20 g to contain 20% of Compound I using XC powder particles. A 15% Compound I solution (concentration of Compound I in this solution could range from 1% Compound I to 15% Compound I) was prepared by adding Compound I (4.00 g) to a 50% acetic acid solution (29.97 g) (concentration of acetic acid solution could range from 50% to 100% acetic acid). Separately, an XC suspension was prepared by adding crystalline FDKP particles (16.00 g) to deionized water (957.33 g) (suspension solids=1.64%) (solids content of the XC suspension could range from 0.5% to 5%). The Compound I solution was then added to the XC suspension and the resulting Compound I XC suspension was spray dried using a Buchi B-290 spray dryer with the conditions shown in Table 3 to produce a 20% Compound I XC powder. Crystalline composite dry powder compositions were also made at 40 wt % and 60 wt % Compound I, using similar process steps for making formulations as described above. Data from these samples are also shown in Table 4 below.

### Preparation of Crystalline (T) Dry Powders Using Compound I

15% powder preparation for a target yield of 2.5 g were made using a Compound I solution (concentration of Compound I in this solution could range from 1% Compound I to 15% Compound I) was prepared by adding Compound I (0.125 g) (Compound I charge could range from 0.025 g to 0.75 g) to a 50% acetic acid solution (0.708 g) (concentration of acetic acid solution could range from 50% to 100% acetic acid). The Compound I solution was added to a T suspension (11.04% solids, 21.51 g) (solids content of the crystalline T suspension could range from 0.5% to 20%) (suspension charge would be adjusted to obtain the desired Compound I charge in the final powder). The Compound I T suspension was spray dried using a Buchi B-290 spray dryer with the conditions shown in Table 3 to produce the desired Compound I T powder.

TABLE 3
Compound I Powder Spray Drying Conditions

| Spray Dryer Parameter | Set Point | |
|---|---|---|
| Inlet Temperature | 170° | C. |
| Aspirator Pump Speed | 90% | |
| Feed Pump Speed | 25% | |
| Nitrogen Flow | 60 | mm |

### Powder Testing

Powders were evaluated for geometric particle size distribution using a Sympatec laser diffraction instrument fitted with a cuvette system. Powders were dispersed in aqueous acetic acid solutions adjusted to pH 4.5 for evaluation. Powders were also evaluated for aerodynamic particle size distribution using an Alberta Idealized Throat Model (AIT). Powders were discharged through the AIT model from Gen 2C cartridges (10 mg cartridge fills) at 4 kPa. HPLC analysis was used to determine Compound I assay of these powders. Data for Compound I powders prepared to date is shown in Table 4.

TABLE 4
Compound I Powder Data

| | | | | | Symaptec | | | |
|---|---|---|---|---|---|---|---|---|
| | | | | | Cuvette Data | | AIT Data | |
| | Target | % | | Compound I | (pH 4.5) | | Avg. | |
| Powder | Yield | Solid | Yield | Assay | x(50) | x(90) | CE | MtF/F |
| Description | (g) | (%) | (%) | (Wt %) | (μm) | (μm) | (%) | (%) |

XC Powders

| | | | | | | | | |
|---|---|---|---|---|---|---|---|---|
| 5% Compound I | 2.5 | 1.37 | 47.6 | 5.53 | 2.05 | 7.09 | 97.1 | 61.6 |
| 10% Compound I | 2.5 | 1.44 | 44.0 | 11.27 | 2.70 | 7.94 | 96.5 | 60.6 |
| 15% Compound I | 2.5 | 1.52 | 56.4 | 17.19 | 2.32 | 7.27 | 98.4 | 63.3 |
| 20% Compound I | 2.5 | 3.12 | 61.6 | 22.06 | 1.95 | 8.44 | 95.5 | 61.6 |
| 20% Compound I | 20 | 2.00 | 66.0 | 21.14 | 2.89 | 10.98 | 95.2 | 76.4 |
| 25% Compound I | 2.5 | 2.42 | 66.4 | 24.31 | 2.70 | 6.41 | 93.9 | 57.2 |
| 30% compound I | 2.5 | 2.56 | 50.5 | 23.03 | 3.15 | 9.85 | 96.0 | 55.5 |
| 40% Compound I | 16 | 1.90 | 75.8 | 42.68 | N/A | N/A | 88.4 | 40.8 |
| 60% Compound I | 16 | 2.00 | 73.1 | 61.48 | N/A | N/A | 83.7 | 50.9 |

T Powders

| | | | | | | | | |
|---|---|---|---|---|---|---|---|---|
| 5% Compound I | 2.5 | 11.19 | 69.2 | 5.13 | 1.30 | 2.86 | 88.0 | 59.7 |
| 10% Compound I | 2.5 | 11.34 | 78.4 | 10.47 | 1.42 | 3.25 | 90.4 | 54.7 |
| 15% Compound I | 2.5 | 11.49 | 90.0 | 15.93 | 1.48 | 4.12 | 83.2 | 47.3 |
| 20% Compound I | 2.5 | 11.65 | 71.6 | 21.63 | 1.49 | 3.80 | 85.6 | 49.0 |
| 25% Compound I | 2.5 | 8.33 | 52.0 | 21.7 | 1.47 | 13.78 | 91 | 49.1 |
| 30% Compound I | 2.5 | 8.59 | 62.0 | 27.83 | 1.38 | 13.18 | 76.5 | 43.6 |

N/A: Data not available

The data illustrates that all XC and T powders comprising Compound I were suitable for pulmonary delivery or for inhalation as shown by the high percent cartridge emptying (CE) data and MtF/F data of the samples tested, which indicate that the powder are easily aerosolizable and can be delivered at high concentrations to the respiratory tract.

### Example 4

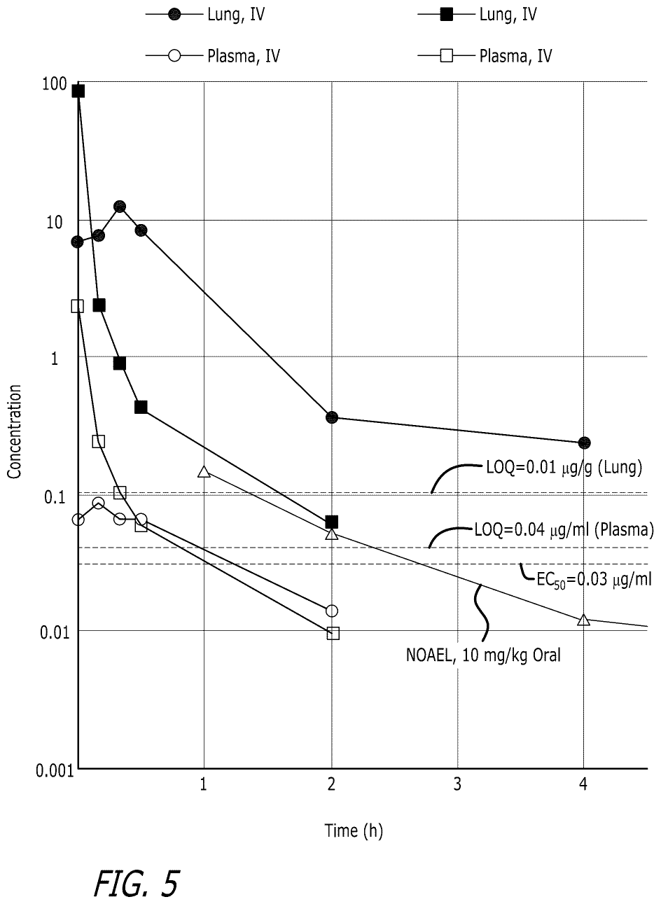

Pharmacokinetic studies in rats with Compound I crystalline composite (XC) dry powders—Insufflation studies were performed in rats to deliver doses of dry powders of Compound I, a protein kinase antagonist. The study was performed to determine the pharmacokinetics profile of Compound I dry powders made by the process as described above following pulmonary insufflation, or intravenous injection of a single dose of the composition administered in solution to male Sprague Dawley (Charles River Laboratories) adult rats, weighing between 200 g and 250 g at the time of the study. Dry powder comprising 22.1 wt % actual drug content were used and tested at 1 mg/ml in solution to deliver a dose of 0.8 mg/kg to 18 rats in each study group. Rats acclimatized and anesthetized prior to experimentation. Lung and blood samples were taken from the rats at 10, 20, 30, 120, and 240 minutes, post drug administration. The samples were analyzed, and the results are presented in FIG. 5.

FIG. 5 illustrates the lung and plasma concentration of Compound I administered by insufflation (circles) and by IV injection. As shown in FIG. 5, a higher content of Compound I was detected in rat lung after insufflation versus IV injection for a longer period of time after administration and more of Compound I remained in lung tissue. The data indicate that insufflation and inhalation Compound I would be more effective in treating disease than delivered by other routes of administration.

### Example 5

### Large Scale Preparation of Lyophilized 20% Nintedanib T Powder:

### Step 1. Preparation of Washed Technosphere Suspension.

Step A. Preparation of polysorbate 80 solution: polysorbate 80 (100.26 g) was slowly added to 900 g of purified water to form a 10% w/w solution.

Step B. Preparation of FDKP solution: Strong ammonia (1280 g) is added to purified water (76.12 kg) under agitation. FDKP (2.2 kg) is added to the ammonia solution under agitation. Mix solution until a clear solution is obtained with no visible undissolved particles. 400 g of the 10% polysorbate solution from step A is added to the FDKP ammonia solution. The resultant solution is mixed until a clear and uniform solution is obtained. The solution is filtered using a Fluorodyne® II filter.

Step C. Preparation of acetic acid/polysorbate 80 solution: A 10.5% acetic acid solution is prepared by adding glacial acetic acid (10.1 kg) to purified water (85.42 kg) with mixing until a clear solution is obtained. To this solution is added polysorbate 80 (480 g) with mixing until a clear solution is obtained. The solution is filtered using a Fluorodyne® II filter.

Step D. Technosphere suspension preparation: A receiver tank is charged with 40 kg of purified water. A Cavitron® high shear mixer is used to feed the filtered solutions from Step B and Step C into the receiver tank. The suspension in the receiver tank is washed using tangential flow filtration (TFF). After washing, 16.34 kg of a technosphere suspension is collected and found to have an average of 8.21% solids.

### Step 2. Preparation of 10% Nintedanib Solution.

A 15% solution of acetic acid was prepared by adding glacial acetic acid (60.02 g) to purified water (340.2 g). A 10% nintedanib solution was prepared by adding nintedanib (46.8 g) to this acetic acid solution.

### Step 3. Preparation of the Nintedanib Inhalation Powder

The 10% nintedanib solution prepared in step 2 was added to a technosphere (T) suspension prepared in a manner analogous to Step 1 above (6.2% solids content, 2580.8 g) with agitation. The resulting suspension is mixed until a uniform and foam-free suspension is obtained. The nintedanib T suspension was then spray dried using a SD micro spray dryer with conditions shown in Table 5. The resultant yield was 145.6 g (72.8%).

TABLE 5
Large scale Nintedanib Inhalation
Powder Spray Drying Parameters

| Spray Dryer Parameter | Setting | |
|---|---|---|
| Inlet Temperature | 175° | C. |
| Outlet Temperature | 85° | C. |
| Drying gas flow | 30 | kg/hr |
| Atomizer gas pressure | 4.1 | bar |
| Atomizer gas flow | 4.8 | kg/hr |
| Heater box temperature | 25.1° | C. |

### Example 6

Toxicology study using the inhalable compositions to test dose tolerance: Inhalable T powders comprising nintedanib as described above in Example 1 were prepared and tested for their toxicologic effect which comprised 4 repeat-dose inhalation studies. A fifth study, a 6-month study in Beagle dogs was also conducted. Special attention was focused on histopathology in the airways of the animals. Additional end points included respiratory/pulmonary function assessment and electrocardiography. Doses derived from the 7-day DRFs were employed in 28-day GLP toxicology studies in rats and dogs. The results from these studies were used to develop initial estimates on the maximum human equivalent doses, and the maximum recommended starting dose for human subjects.

Target and achieved doses were reported in terms of lung-deposited dose per unit body weight (mg/kg). The deposited doses were calculated from the presented doses using deposition factors of 10% (rats) and 25% (dogs). The dog was chosen as the non-rodent species for the toxicology program as it was the most sensitive species used in previously completed nintedanib nonclinical studies (Australian Therapeutic Goods Administration. Australian Public Assessment Report for Nintedanib esilate. auspar-nintedanib-esilate-160208. 2016.) In that program, dogs could not tolerate the GI effects of oral nintedanib so non-human primates were ultimately selected as the second species. GI and systemic effects were studied and the results showed less severe outcomes in the animals.

There were no observed adverse effect levels (NOAELs, expressed as deposited dose per unit body weight) from the rat and dog studies (2.64 and 0.85 mg/kg/day, respectively). Estimates of the maximum human dose (MHD) supported by the 28-day inhalation studies were calculated based on deposited dose per unit body weight and deposited dose per unit lung weight and the appropriate minimum safety margin (10 for rats and 6 for dogs). The four estimates of MHD ranged from 8.5 mg/day (dogs, dose/body weight) to 44 mg/day (rats, dose/lung weight). Therefore, the MHD for nintedanib inhalation dry powder is at least 8.5 mg/day of nintedanib for lung delivery.

Local Tolerance-tolerance studies were conducted using nintedanib by insufflation in animal airways were collected during the 28-day inhalation studies in rats and dogs. In rats, main study mid- and high-dose animals (and one low-dose animal) developed minimal epithelial degeneration in the tracheal epithelium, primarily at the level of the carina. This finding fully resolved following a 14-day recovery in all low- and mid-dose animals but persisted minimally in 2/5 high-dose females (2/10 M+F combined). This minimal tracheal change was considered non-adverse.

Additionally, main-study animals in all dose groups developed squamous metaplasia in the laryngeal epithelium. Squamous metaplasia is a common response by the laryngeal epithelium in inhalation studies. It is a response to physical or other factors that results in a more resistant type of epithelium in this susceptible site; it is considered an adaptive defense mechanism. In this study, the change was minimal, there was no evidence of remarkable cellular atypia or extension of this change into other areas of the larynx or respiratory tract, and the response was not observed in the recovery animals. This common change in the rat is typically reversible and is generally considered to be non-adverse. Its presence in inhalation studies in the rat is common and is a poor predictor of any similar response in primates or other species.

In dogs, inhalation of nintedanib powder at all examined doses of the 28-day study (0.18-0.85 mg/kg) led to histologic changes limited to the respiratory tract. Changes in the pulmonary tract included a low incidence of minimal centriacinar mixed cell inflammation, increased alveolar macrophages, and bronchial epithelial degeneration. The incidence of elevated alveolar macrophages climbed in a dose dependent manner. This is a common non-adverse finding in inhalation studies and was resolved at recovery. The incidence of minimal mixed cell inflammation in dogs receiving the mid- and high-doses of 0.43 and 0.85 mg/kg was slightly higher than in historical controls. This increased incidence persisted into recovery. Given the minimal nature of the change, it was deemed non-adverse. Minimal degeneration of bronchial epithelium developed in some dogs exposed to nintedanib inhalation powder, with incidence increasing in a dose-dependent manner. Incidence was notably decreased at recovery, with degenerative changes persisting focally in only a few animals. Due to the minimal nature and evidence of resolution at recovery, this finding was deemed non-adverse.

Nasal changes were limited to the squamous epithelium of Level 1 and the respiratory epithelium of Level 2. Minimal mixed cell infiltrate was found at the ventral aspect of the nares of Level 1 in dogs at all doses of nintedanib inhalation powder. This change was mostly resolved at recovery, with only one dog affected. Two female dogs receiving the mid dose developed moderate degeneration of the respiratory epithelium, with one dog developing minimal ulceration and mild mixed cell inflammation. These findings at Level 2 were resolved in recovery. All nasal findings were deemed non-adverse.

In studies with both rats and dogs, the powder burden in the lung (deposited dose/lung weight, mg/g LW) was relatively high. The dose range administered to rats, 0.44-2.64 mg/kg BW, corresponds to a powder burden of 0.073-0.44 mg/g LW for a standard 250 g rat with 1.5 g lungs [Tepper 2016]. Similarly, the inhaled dose range for dogs, 0.18-0.85 mg/kg BW is 0.016-0.077 mg/g LW for a 10 kg dog with 110 g lungs [Tepper 2016].

The maximum human dose (MHD) supported by the 28-day inhalation studies were calculated based on deposited dose per unit body weight and deposited dose per unit lung weight at the appropriate minimum safety margin (10 for rats and 6 for dogs). The four estimates of MHD ranged from 8.5 mg/day (dogs, dose/body weight) to 44 mg/day (rats, dose/lung weight). Therefore, the MHD for nintedanib inhalable powder (or NIP) is 8.5 mg/day of nintedanib. In one embodiment for human use, the calculated nintedanib inhalable powder dose for lung delivery is from about 1 mg to 10 mg (wt %) in the formulation. In one embodiment, about 2 mg to 8 mg per day of nintedanib, equivalent to 0.033-0.133 mg/kg BW and 0.002-0.008 mg/g LW for a 60 kg person with 1000 g lungs [Tepper 2016], is administered per inhalation dose. Higher doses can be delivered by inhalation to a patient depending on the patient's needs.

By delivering nintedanib directly to the lung, inhaled nintedanib powder provides local therapeutic concentrations in the lungs with a reduction in the systemic concentration and its associated adverse effects can be minimized when administered to human subjects at reduced quantities (less than the 300 mg oral daily dosage, 150 mg twice daily capsules, currently the standard of care).

Inhaled nintedanib powder was well-tolerated by both rats and dogs at doses providing adequate safety margins for the human doses. All doses tested by inhalation in rats and dogs yielded lung concentrations greater than ˜50 ng/g when measured 24 hours after the final dose.

Histopathologic changes in tissues were limited to the airways of the test animals, but this may be related to the relatively heavy powder burden in the lung. The estimated lung burden at the high end of the clinical dose range, 0.008 mg/g LW, is half that at the low end of the dog dose range (0.016 mg/g LW) and 10% of the high end. The nonclinical data indicate that human doses should be safe and tolerable.

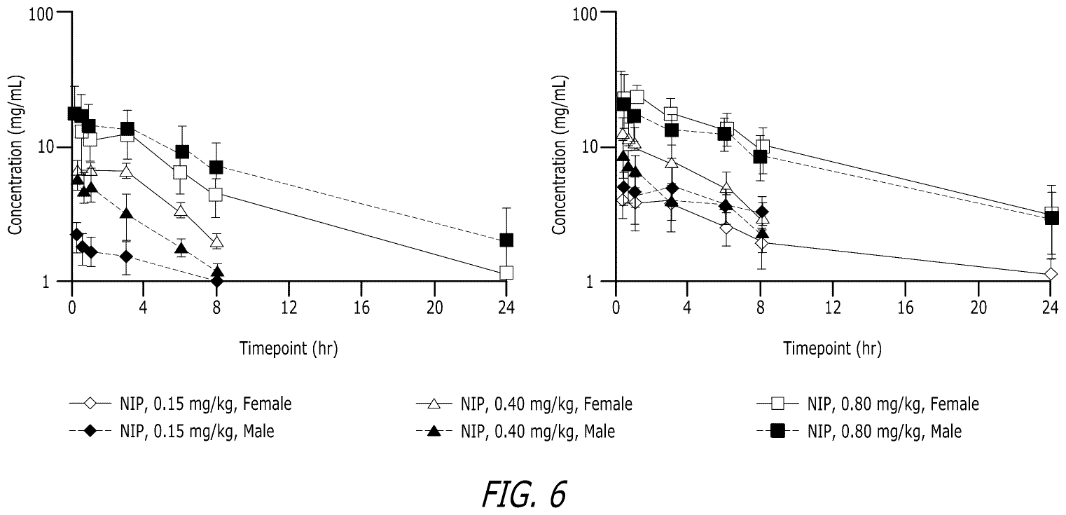

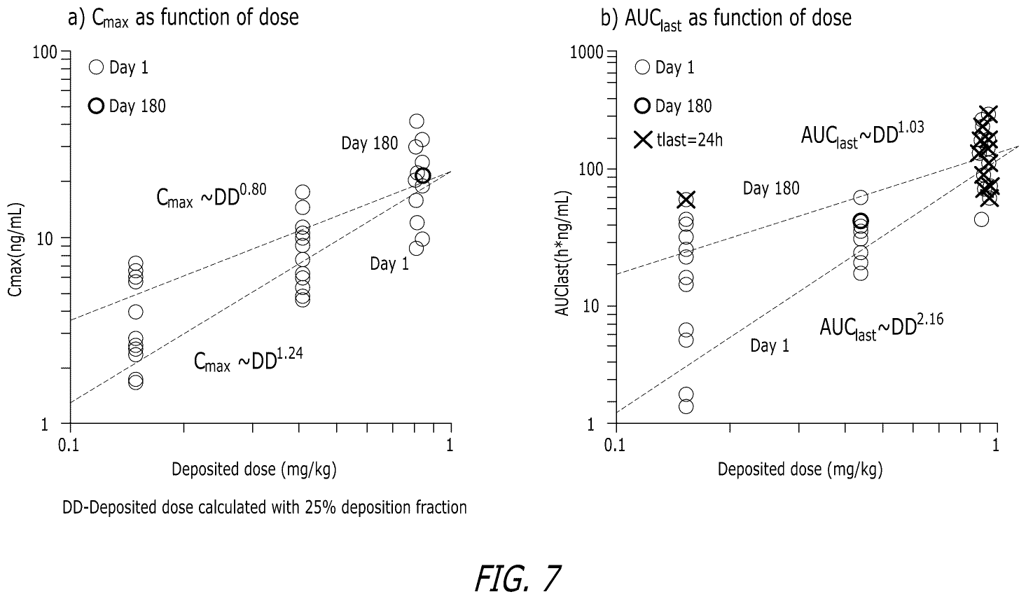

Six month dog study—A six-month chronic toxicity study in male and female beagles was conducted. 32 beagle dogs were randomly assigned to 4 groups (4 male and 4 female dogs per group): filtered air control, or nintedanib inhalation powder at nominal low (0.15 mg/kg), mid (0.4 mg/kg), or high (0.8 mg/kg) doses once a day for 180 consecutive days via face-mask inhalation. Blood was collected after exposures on day 1 and day 180 (20 min, 40 min, 1 hr, 3 hr, 6 hr, 8 hr, and 24 hrs), as well as predose on day 180 for toxicokinetics. Lungs were assayed at sacrifice (24 hours after the final exposure). Additional endpoints included clinical observations, ophthalmology exams, body weight and food consumption, organ weights, clinical pathology, electrocardiology, pulmonary function, and gross and microscopic pathology. Cₘₐₓ occurred at 20 or 40 minutes after exposure and a clear dose response was evident in the mean plasma profiles on day 1 and day 180 (FIG. 6). Some concentrations in the low- and mid-dose groups were particularly low on day, resulting in super-proportional dose response on day 1 and weaker dependence on day 180 (FIG. 7); this generated apparent dose-dependence in accumulation ratios (Day 180/Day 1). The geometric mean accumulation ratio for the treated animals was 1.62 for Cₘₐₓ and 2.12 for AUCₗₐₛₜ. The beagle dogs showed no associated adverse events during the study.

### Example 7

### a Phase 1, Randomized, Double-Blind, Placebo-Controlled, Single- and Multiple-Ascending Dose Study to Evaluate the Safety, Tolerability, and Pharmacokinetics of Nintedanib Inhalation Powder (NIP) in Healthy Volunteers.

The study was designed to evaluate the safety and tolerability of single and multiple-ascending doses of nintedanib inhalation powder (NIP) versus placebo in healthy adult participants. First, the study was designed to evaluate the pharmacokinetics (PK) of NIP, following single and multiple-ascending doses in healthy adult participants. A second study was intended to evaluate the effect of NIP versus placebo on pulmonary function immediately post-dose.

### Methodology:

A Phase 1, first-in-human (FIH), randomized, double-blind, placebo-controlled study of nintedanib inhalation powder (NIP) in healthy adult participants was conducted. The study was conducted in 2 parts: (Part A) a single dose, dose-escalation in 3 cohorts, followed by (Part B), a multiple dose, dose-escalation in 2 cohorts. In each cohort, participants were allocated in a randomized and double-blind manner at a ratio of 3:1 to receive NIP or placebo via inhalation. The study was planned to investigate the safety, tolerability, and PK of NIP.

### Test Product, Dose, and Mode of Administration:

NIP is a dry powder nintedanib formulation for oral inhalation (nintedanib containing Technosphere® particles). NIP was supplied in 2 mg cartridges containing 2 mg nintedanib dry powder for oral inhalation. NIP was administered in Part A as a single dose of 2 mg, 4 mg, or 8 mg in Cohorts A1 (1 cartridge), A2 (2 cartridges), or A3 (4 cartridges); respectively. NIP was administered in Part B as a twice daily dose (in the morning and in the evening) of 2 mg or 4 mg in Cohorts B1 (1 cartridge) and B2 (2 cartridges) respectively. Placebo control was made up of the same Technosphere® particles as NIP but without the active pharmaceutical ingredient (API) nintedanib. Placebo control was administered in Part A as a single dose in Cohorts A1 (1 cartridge), A2 (2 cartridges), or A3 (4 cartridges) and in Part B as a twice daily dose (in the morning and in the evening) in Cohorts B1 (1 cartridge) and B2 (2 cartridges).

In Part A pf the study a single dose administered on Day 1. In Part B of the study, administration of nintedanib was twice daily dose for 7 days.

### Pharmacokinetics:

The following pharmacokinetic (PK) parameters were calculated based on plasma concentrations of nintedanib from the plasma concentration-time data as appropriate:

Maximum observed plasma drug concentration (Cₘₐₓ), time to reach Cₘₐₓ (Tₘₐₓ), minimum observed plasma drug concentration over a dosing interval (Cₘᵢₙ), observed plasma drug concentration at 12 hours after dose administration (C₁₂), time to reach Cₘᵢₙ (Tₘᵢₙ), average concentration (C_{avg}), last quantifiable concentration (Cₗₐₛₜ), time of Cₗₐₛₜ (Tₗₐₛₜ), area under the plasma concentration-time curve from time 0 to 12 hours (AUC₀₋₁₂), area under the plasma concentration-time curve from time 0 to the last measurable plasma concentration (AUC₀₋ₜ), area under the plasma concentration-time curve from time 0 to infinity (AUC_{0-∞}), area under the plasma concentration-time curve over a dosing interval (AUCₜₐᵤ), AUC extrapolated from the last plasma drug concentration to infinity in percent of the total AUC (fₑₓₜ), apparent total plasma clearance after extravascular administration (CL/F), apparent volume of distribution based on terminal phase after extravascular administration (V_{z}/F), apparent first-order terminal elimination rate constant (kₑₗ), terminal elimination phase half-life (t_{1/2}).

### Safety:

The following safety and tolerability parameters following single and multiple-ascending doses of NIP versus placebo in 40 healthy adult participants were assessed:

Incidence of inhaled intolerability (prevalence of cough, dyspnea, bronchospasm, and dysgeusia); Incidence of participants with reductions in forced expiratory volume in 1 second (FEV₁)≥15% from baseline at any time post-dose; Incidence of participants with reductions in FEV₁≥15% following administration of a dose of study drug, compared to the corresponding pre-dose measurement; Incidence, severity, duration, relationship to study drug, and outcome of treatment-emergent adverse events (TEAEs); Incidence, severity, duration, relationship to study drug, and outcome of serious adverse events (SAEs); Incidence of abnormal, clinically significant vital signs (heart rate, blood pressure, respiratory rate, oxygen saturation rate, and body temperature); Changes from baseline in liver enzymes and bilirubin; Changes from baseline in coagulation parameters.

### Pharmacokinetics Analysis:

All noncompartmental PK analyses were performed using standard techniques as implemented in PKanalix® 2024R1. Summary statistics were prepared and dose proportionality statistical assessment was performed using R Version 4.1.3. The PK population consisted of all participants who received at least 1 dose of NIP and had a sufficiently evaluable plasma concentration-time profile of nintedanib to have allowed determination of at least 1 PK parameter.

Noncompartmental PK parameters were calculated for each individual and study day. Plasma PK parameters for nintedanib were estimated from plasma concentration-time data for each cohort after inhaled dose administration. Dose proportionality of nintedanib plasma concentration data was assessed using the power model approach. Descriptive statistics included sample size, arithmetic mean, standard deviation (SD), coefficient of variation expressed as a percent (% CV), geometric mean, geometric mean % CV, minimum, median, and maximum. Pharmacokinetic parameter values were used for the calculation of summary statistics without rounding.

### Safety Analysis:

All safety analyses were performed on the Safety Population. Values for all safety variables were to be listed by participant and date. Adverse events were mapped to a Medical Dictionary for Regulatory Activities (MedDRA) Version 27.0 preferred term and system organ classification. If a participant experienced multiple events that mapped to a single preferred term, the greatest severity grade according to the World Health Organization (WHO) Toxicity Criteria as assessed by the Investigator, and strongest Investigator assessment of relation to study drug was to be assigned to the preferred term for the appropriate summaries. Participants with reported adverse events (AEs) relating to inhaled intolerability, which included cough, bronchospasm, dyspnea, and dysgeusia, were summarized by treatment. Descriptive summaries of selected (quantitative) clinical laboratory results and changes from study baseline were presented by treatment group and study visit. Laboratory toxicities were programmatically graded by severity using Common Terminology Criteria for Adverse Events (CTCAE) Version 5.0. Vital signs and corresponding changes from study baseline were summarized by treatment group, position, and visit using descriptive statistics. Participants with abnormal clinically significant vital signs were summarized by treatment. ECGs and corresponding changes from study baseline were summarized by treatment group and visit. Within treatment changes in FEV₁ were summarized.

### Pharmacokinetics

In general, nintedanib plasma concentrations increased with increasing dose after single dose administration of NIP (2 mg to 8 mg). Substantial variability in observed nintedanib plasma concentrations and calculated exposures was observed between participants after both single- and multiple-dose administration and within participants based on multiple-dose administration. Nintedanib was rapidly absorbed with maximum concentrations observed at the first measured sample immediately (within a few minutes) after the inhaled dose administration.

Terminal elimination half-life was similar between dose groups after single-dose administration in Part A with geometric mean values ranging between 10.3 hours and 14.5 hours. The calculated half-life values from Part B were generally shorter than those from Part A, most likely due to the differing time range of data for calculation (24 hours after dose administration for Part A and 12 hours after dose administration for Part B) and the multi-phasic nature of the plasma concentration versus time profiles.

In Part A, Cₘₐₓ concentrations increased as dose increased: geometric mean (% CV) Cₘₐₓ values of 4.10 ng/ml (103.9%) for the 2-mg dose group, 6.84 ng/ml (107.4%) for the 4 mg dose group, and 27.2 ng/ml (45.6%) for the 8-mg dose group. In Part B, for both Day 1 and Day 7, Cₘₐₓ increased for the 2-mg dose but not the 4-mg dose: geometric mean (% CV) Cₘₐₓ values on Day 1 of 2.15 ng/ml (128.9%) for the 2-mg dose group and 17.9 ng/ml (28.9%) for the 4-mg dose group with values on Day 7 of 8.43 ng/ml (24.4%) for the 2-mg dose group and 11.4 ng/ml (42.7%) for the 4-mg dose group. The lack of increase in the concentration from Day 1 to Day 7 in the 4-mg group was presumed to be due to the small sample size and high variability. On Day 7 in Part B, the calculated geometric mean AUCₜₐᵤ was 8.38 h×ng/mL for the 2-mg dose group and 14.9 h×ng/ml for the 4-mg dose group. Calculated Day 7 geometric mean CLₛₛ/F was 239 L/h for the 2-mg dose group and 269 L/h for the 4-mg dose group, and geometric mean V/F was 5,580 L for the 2-mg dose group and 5,310 L for the 4-mg dose group. The geometric mean accumulation AUCₜₐᵤ ratios on Day 7 were 4.93 for the 2-mg dose group and 1.45 for the 4-mg dose group, and the geometric mean Cₘₐₓ accumulation on Day 7 was 3.91 for the 2-mg dose group and 0.637 for the 4-mg dose group. Statistical dose proportionality was not demonstrated for either single-dose or multiple-dose administration in this study.

### Safety Characteristics

Based on the fact that all TEAEs reported in the study were mild and no SAEs, severe TEAEs, TEAEs leading to discontinuation or dose adjustments, or deaths were reported, then the study drug is considered safe and well tolerated in this adult healthy population and over the study duration. All TEAEs reported in the study were considered related to the study treatment. Only 2 types of AEs were reported, and both were respiratory-related: cough and FEV₁ reduction of ≥15%, and they did not have any associated signs or other symptoms. More specifically, no GI (e.g., diarrhea, nausea, vomiting, abdominal discomfort) or neurologic (e.g., headache, fatigue) AEs were reported in this study.

None of the abnormalities in vital signs that were deemed “clinically significant” as defined in the protocol showed any treatment-related trends. No TEAEs or SAEs related to clinically significant vital signs abnormalities were reported. No clinically significant ECG abnormalities or TEAEs related to ECG abnormalities were reported. The incidence of ≥15% reduction in FEV₁ from baseline and pre-dose were observed within 30 minutes of NIP administration (Dose 1 only). No treatment-related trends were observed. Neither cough nor ≥15% reduction in FEV₁ worsened with increased or ongoing dosing. Cough was more common after the first cartridge intake regardless of the overall dose. No clinically significant abnormal laboratory results were reported. No TEAEs related to changes from baseline in liver enzymes, bilirubin, or coagulation parameters were reported.

### Example 8

A Randomized, Active-Controlled, Open-Label, Clinical Trial Study of the Efficacy and Safety of Nintedanib Inhalation Powder (NIP) in Patients with Idiopathic Pulmonary Fibrosis (IPF) with an Open-Label Extension

The clinical trial is made up of two studies and is conducted using approximately 250 study centers located internationally. The study is a randomized controlled treatment (RCT) period to assess the short-term safety and efficacy of a range of doses, to confirm an optimal dose of NIP in patients with IPF over at least 16 weeks and up to 48 weeks. The objective of this study is an open-label extension (OLE) to assess long-term safety and tolerability of NIP in patients with IPF.

Another objective of the RCT study is to assess and confirm efficacy, safety and tolerability of NIP in patients with IPF over at least 36 weeks and up to 48 weeks.

A further objective of the study is an OLE study to assess long-term safety and tolerability of NIP in patients with IPF.

### Study Design and Methodology:

This clinical trial is designed as two open-label, active controlled studies, each with an OLE, conducted in an operationally and inferentially seamless manner. The clinical trial aims to compare the efficacy and safety of NIP versus oral nintedanib for the treatment of IPF.

The purpose of the RCT study is to evaluate the safety and tolerability of different doses of NIP and potentially to demonstrate non-inferior efficacy compared to the active control (oral nintedanib) in patients with IPF, in modifying the rate of decline of forced vital capacity (FVC). The study involves 100 participants, with about 25 participants randomized to each of the 4 study arms.

The study RCT period will last 16-48 weeks. During this period participants will be randomized to 1 of 3 NIP dosing regimens (NIP 2 mg TID, NIP 4 mg BID, or NIP 4 mg TID; N=25 each) or oral nintedanib (N=25). Participants randomized to oral nintedanib will receive the approved dose of 150 mg taken orally twice a day (BID). Any modification to the oral nintedanib dose will be managed by the participant's healthcare provider and the reasons for such changes will be documented.

The study RCT period is followed by the OLE period of the study where all eligible participants will continue dosing with NIP (if they were on 1 of the 2 NIP 4 mg cohorts in the RCT) or initiate treatment with NIP (if they were previously on oral nintedanib or the lower NIP dose).

The Data and Safety Monitoring Board (DSMB) will review safety data at predefined enrollment milestones, specifically after the randomization of 15, 30, 50, 75, and 100 participants. The decision to proceed with subsequent study enrollment, as planned, will be based on DSMB review of unblinded data from the first 75 participants who have received at least 1 dose of the study drug.

The primary efficacy endpoint of the study is the annualized rate of decline in FVC, over at least 16 weeks. The secondary efficacy endpoint is time to overall mortality or disease progression over 16 to 48 weeks.

One aspect of the study is to confirm efficacy, safety, and tolerability of NIP at an optimal dose(s) in patients with IPF having a sample size expands to an additional ˜780 participants, with ˜450 randomized to the single selected dose of NIP based on the study results and ˜225 randomized to the active control arm of oral nintedanib. The control arm will be supplemented using Bayesian dynamic borrowing, targeting an additional effective sample size of up to 225 historical participants who received oral nintedanib. Two interim analyses will be conducted during this part of the study.

The RCT period will be 36 weeks to 48 weeks. Participants will initially be randomized to 1 of 3 NIP dosing regimens or oral nintedanib. Once a dose is selected, the remaining participants entering the subsequent RCT study will be randomized 2:1 (NIP to oral nintedanib). Participants are randomized to oral nintedanib of a standard dose (150 mg BID), unless otherwise prescribed by their healthcare provider in case of intolerability, and any changes to dosing will be documented.

The RCT period is followed by the OLE period, where all eligible participants will continue dosing with NIP (if they were on 1 of the 2 NIP 4 mg cohorts in the RCT) or initiate treatment with NIP (if they were previously on oral nintedanib or the lower NIP dose of 2 mg TID). Participants will continue in the OLE period on NIP at a dose of 4 mg BID or TID, until a single NIP dose is selected based on the study, at which time all study participants in the OLE will transition to that single dose.

The DSMB will review safety data at predefined enrollment milestones, specifically after the randomization of 45, 90, 140, 200, 300, 450, and 600 participants. Subsequent DSMB reviews will occur quarterly. The DSMB, with the support of an independent statistician, will also perform 2 interim analyses.

The primary endpoint of this part of the study is the annualized rate of decline in FVC (mL), with a primary analysis of FVC using a Bayesian linear mixed effects model with random intercepts and slopes, adjusting for age and gender. The key secondary endpoint is pulmonary exacerbation, adjusted for the Gender-Age-Physiology (GAP) score.

### Dosing of NIP

Study participants randomized to the 2 mg TID NIP study arm will receive a single cartridge of NIP (2 mg) three times a day (TID), about 6 hours apart during waking hours. Study participants randomized to the NIP 4 mg BID study arm will receive a single cartridge of NIP (2 mg) twice a day (about 12 hours apart) for one week, followed by 2 cartridges (each 2 mg each for a total dose of 4 mg) twice a day, about 12 hours apart, starting on Day 8.

Study participants randomized to the NIP 4 mg TID study arm will receive a single cartridge of NIP (2 mg) three times a day (about 6 hours apart during waking hours) for 1 week. followed by 2 cartridges (each 2 mg for a total dose of 4 mg) three times a day, about 6 hours apart during waking hours, starting on Day 8.

All OLE study participants will receive NIP at a dose of either 4 mg BID or TID, administered as two 2 mg cartridges twice or three times a day, respectively. Participants will continue to receive NIP 4 mg BID or TID for the remainder of the OLE study, until a decision is made on which single NIP dose will be developed further, at which time all study participants in the OLE will be changed to that single dose level.

### Dosing of NIP in Subsequent Study:

Study participants will receive the same dosing as in the initial study with the randomization schema of 1:2:2:1 at screening until the results of the study become available. Once a single dose level is selected, the randomization schema will change to 2:1 (selected NIP dose regimen: control). Participants who were in the oral nintedanib arm and those on the 2 mg TID will initiate the OLE with NIP 4 mg BID or TID, receiving 2 cartridges (each 2 mg for a total dose of 4 mg) by inhalation twice or three times a day. After the determination of tolerability of doses in the first study, a dose is chosen, then all OLE study participants will be switched to the selected dose.

### Dosing of Oral Nintedanib (Active Control Arm) in Both Parts of RCT Periods:

Study participants randomized to the active control study arm (oral nintedanib) will receive one capsule of oral nintedanib (100 mg) twice a day (about 12 hours apart) for one week, followed by one capsule of oral nintedanib (150 mg) twice a day, about 12 hours apart, starting on Day 8.

### Dose Adjustments:

In the event of intolerance to inhaled NIP, the study participant, in consultation with their healthcare provider, may decrease the dose to 2 mg (one cartridge) inhalation BID or TID, if needed. Participants have the option to re-up-titrate to the 4 mg BID or TID dosing in consultation with the investigator. Similarly, for the active control study arm: in the event of intolerance to oral nintedanib, the study participant, in consultation with their healthcare provider/investigator, may decrease the dose from ISO mg to 100 mg orally BID, if needed. Participants have the option to re-up-titrate to the 150 mg BID dose in consultation with the investigator. There are no limits on the number of times a study participant may re-up-titrate to the target dose for either the inhaled or oral study arm intervention. This is a clinical decision between the study participant and their healthcare provider/investigator. These down-titrations and up-titrations will be documented during the study.

If the lower dose (2 mg) is not tolerated, the study participant may stop the study drug per guidance from the Principal Investigator (PI). Re-challenge with the 2 mg dose may be done per the PI's clinical judgement.

### Criteria for Evaluation:

The primary (efficacy) endpoint is the annualized rate of decline in FVC, measured in milliliters over at least 16 weeks. The secondary endpoint includes time to overall mortality or disease progression (defined as a ≥10% absolute decline from baseline in FVC percent predicted, adjudicated respiratory-related non-elective hospitalization, adjudicated Acute Exacerbation-IPF, or lung transplantation), over 16-48 weeks; Change from baseline in Living with Pulmonary fibrosis (L-PF) Total score at Week 16; Change from baseline in L-PF symptom dyspnea domain at Week 16; Change from baseline in L-PF symptom cough domain at Week 16.

PK parameters will be calculated for all study participants, regardless of their study arm, based on plasma concentrations of nintedanib as data permits. Maximum plasma concentration (Cₘₐₓ); Time to maximum concentration (tₘₐₓ); Terminal elimination half-life (t_{1/2}); Area under the plasma concentration-time curve (AUC) from time zero (from the start of inhalation time) to the last measurable concentration (AUC₀₋ₜ); AUC from time zero (time of first inhalation) to infinity (AUC_{0-∞}); Apparent terminal elimination rate constant (Kₑₗ); Apparent total body clearance (CL/F); Apparent volume of distribution during the terminal phase (Vz/F).

Events of bronchospasm (e.g., TEAE immediately after inhalation of wheezing or chest tightness); Changes (mL) in FEV₁ from Baseline to Week 16; Rate of study drug discontinuations; Rate of study drug dose reductions; Rate of TEAEs (treatment-emergent AEs); Rate of TRAEs (treatment-related AEs); Rate of SAEs.

Change from baseline in L-PF symptom fatigue domain at Week 16; Change from baseline in L-PF Impact score at Week 16; Change from baseline in VAS score cough severity at Week 16; Change from baseline in EQ-SD-5L score at Week 36; Time to overall mortality through 16-48 weeks; Time to respiratory-related hospitalization or mortality through 16-48 weeks; Time to IPF progression through 16-48 weeks, as defined by a decline in FVC percent predicted of 10% or greater or death; Change in the King's Brief Interstitial Lung Disease questionnaire from baseline to Week 16; FVC percent predicted change from baseline over 16-48 weeks. Study results are analyzed for the annualized rate of decline in FVC, measured in milliliters over 36-48 weeks. Study results are also analyzed for the time to overall mortality or disease progression (defined as a ≥10% absolute decline from baseline in FVC percent predicted, adjudicated respiratory-related non-elective hospitalization, adjudicated Acute Exacerbation-IPF, or lung transplantation) over 36-48 weeks; Change from baseline in Living with Pulmonary Fibrosis (L-PF) Total score at Week 36; Change from base line in L-PF symptom dyspnea domain at Week 36; Change from baseline in L-PF symptom cough domain at Week 36.

Patients in the study are also monitored for frequency of events of bronchospasm (e.g., TEAE immediately after inhalation of wheezing or chest tightness); Changes (mL) in FEV₁ from Baseline to Week 36; Rate of study drug discontinuations; Rate of study drug dose reductions; Rate of TEAEs; Rate of TRAEs (treatment-related AEs); Rate of SAEs.

Change from baseline in L-PF symptom fatigue domain at Week 36; Change from baseline in L-PF Impact score at Week 36; Change from baseline in VAS score cough severity at Week 36; Change from baseline in EQ-5D-SL score at Week 36; Time to overall mortality through 36-48 weeks; Time to respiratory-related hospitalization or mortality through 36-48 weeks; Time to IPF progression through 36-48 weeks, as defined by a decline in PVC percent predicted of 10% or greater, or death; Time to first investigator-reported acute IPF exacerbation through 36-48 weeks; Time to IPF-specific mortality through 36-48 weeks; Change in the King's Briefing Interstitial Lung Disease questionnaire at Week 36; FVC percent predicted change from baseline over 36-48 weeks; Change in oxygen requirements at rest from baseline over 36-48 weeks; Change from baseline in Gastrointestinal Symptom Rating Scale (GSRS) at Week 36; Change from baseline in Functional Assessment of Chronic Illness Therapy-Diarrhea (FACIT-D, Diarrheas sub-domain) at Week 36; Change in use of anti-diarrheal drugs from baseline over 36-48 weeks.

Comparisons to the active control (oral nintedanib) to assess for preliminary efficacy will be made for each NIP dose based on descriptive analyses. For the test of non-inferiority versus active control (oral nintedanib), a total sample size of ˜780 subsequent study participants of which ˜675 participants are randomized 2:1 to the selected NIP dose or oral nintedanib (˜450 on the single selected NIP dose, ˜225 oral nintedanib) will provide 80% power with one-sided alpha=0.025 to detect a treatment difference between the NIP and oral nintedanib arms in the annualized rate of decline in FVC in mL using a non-inferiority margin of 50 mL, assuming a common standard deviation in both groups of 225 mL and no difference between the NIP and oral nintedanib study arms.

The primary analyses will be conducted using a Bayesian linear mixed effects model which incorporates dynamic borrowing to augment the sample size randomized to oral nintedanib with historical estimates from the TOMORROW, INPULSIS-1 and INPULSIS-2 trials. The dynamic borrowing will be tuned so that the maximum effective borrowed sample size does not exceed 225 oral nintedanib participants worth of additional information.

The response variable is observed FVC volume (in mL), including baseline. The model will estimate an intercept term and fixed effects for stratification variables, a NIP-specific (for the single selected NIP dose) additive intercept adjustment, a slope, and a NIP-specific additive slope adjustment. Random effects for participant-specific intercepts and slopes will be specified. Hypothesis testing will be conducted on the difference in slopes of the oral nintedanib and NIP groups, i.e., the NIP-specific additive slope adjustment.

An adaptive design will include 2 interim analyses with prespecified rules for trial futility at the first interim analysis and potential sample size re-estimation at the second interim analysis. Adaptive decision rules and type I error control will be demonstrated via simulation with details provided in an Adaptive Design Report appendix to the SAP.

Safety endpoints, including bronchospasm events, FEV₁ changes, TEAEs, TRAEs, SAEs, study drug discontinuations, study drug dose reductions, clinical laboratory assessments, and vital signs, will be listed and summarized, by cohort and randomized treatment, using descriptive statistics.

### Example 9

A 55 year old male subject with IPF is treated with a single cartridge of NIP (2 mg) three times a day (TID) for 16 weeks. The subject experiences a rate of decline in FVC of less than 5%, the only AE reported is cough and they did not have any other associated signs or other symptoms.

A 47 year old female subject with IPF is treated with a single cartridge of NIP (2 mg) two times a day (BID) for 1 week, followed by 2 cartridges of NIP (each 2 mg for a total dose of 4 mg) BID for 31 weeks. The subject experiences a rate of decline in FVC of less than 10%, the only AE reported is FEV₁ reduction of ≥15% and they did not have any other associated signs or other symptoms.

A 50 year old male subject with IPF is treated with a single cartridge of NIP (2 mg) three times a day (TID) for 1 week, followed by 2 cartridges of NIP (each 2 mg for a total dose of 4 mg) TID for 47 weeks. The subject experiences a rate of decline in FVC of less than 10%, the only AE's reported are cough and FEV₁ reduction of ≥15% and they did not have any other associated signs or other symptoms.

The preceding disclosures are illustrative embodiments. It should be appreciated by those of skill in the art that the devices, techniques and methods disclosed herein elucidate representative embodiments that function well in the practice of the present disclosure. However, those of skill in the art should, in light of the present disclosure, appreciate that many changes can be made in the specific embodiments that are disclosed and still obtain a like or similar result without departing from the spirit and scope of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope otherwise claimed. No language in the specification should be construed as indicating any non-claimed element essential to the practice of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Many embodiments are described herein, including the best mode known to the inventors for carrying out the invention. Of course, variations on the embodiments will become apparent to those of ordinary skill in the art upon reading the foregoing description. The inventor expects those of ordinary skill in the art to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments so claimed are inherently or expressly described and enabled herein.

Furthermore, numerous references have been made to patents and printed publications throughout this specification. Each of the above cited references and printed publications are herein individually incorporated by reference in their entirety.

Further, it is to be understood that the embodiments disclosed herein are illustrative of the principles of the present invention. Other modifications that may be employed are within the scope of the invention. Thus, by way of example, but not of limitation, alternative configurations may be utilized in accordance with the teachings herein. Accordingly, the present invention is not limited to that precisely as shown and described.

We claim:

1. An inhalable pharmaceutical composition, comprising:
a dry powder comprising diketopiperazine particles and a therapeutically effective dose of nintedanib or Compound I having the formula: 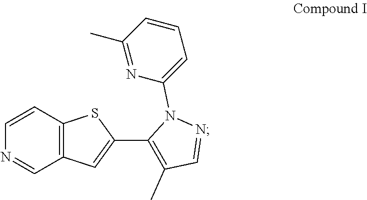 [SMILES: Cc1cccc(-n2ncc(C)c2-c2cc3cnccc3s2)n1]
or a pharmaceutically acceptable salt thereof and optionally, one or more pharmaceutically acceptable carriers and/or excipients.

2. The inhalable pharmaceutical composition of claim 1, wherein the therapeutically effective dose is in an amount of up to about 50 mg of nintedanib or Compound I and the one or more pharmaceutically acceptable carriers and/or excipients.

3. The inhalable pharmaceutical composition of claim 1, wherein the one or more pharmaceutically acceptable carrier and/or excipient is a surfactant, an amino acid, or a phospholipid.

4. The inhalable pharmaceutical composition of claim 1, wherein the diketopiperazine is of the formula: 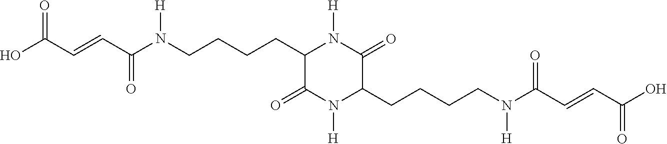 [SMILES: O=C(O)/C=C/C(=O)NCCCCC1NC(=O)C(CCCCNC(=O)/C=C/C(=O)O)NC1=O]
in crystalline, or microcrystalline particle form.

5. The inhalable pharmaceutical composition of claim 1, wherein the therapeutically effective dose of nintedanib or a pharmaceutically acceptable salt thereof, or Compound I or a pharmaceutically acceptable salt thereof, ranges from about 1 mg to about 50 mg in the dry powder composition.

6. The inhalable pharmaceutical composition of claim 1, wherein the dry powder composition is an amorphous powder.

7. The inhalable pharmaceutical composition of claim 1, wherein the dry powder comprises one or more pharmaceutically acceptable carriers and/or excipients is selected from lactose, mannose, sucrose, mannitol, trehalose, sodium citrate, trisodium citrate, zinc citrate, glycine, L-leucine, isoleucine, trileucine, sodium tartrate, zinc tartrate, methionine, vitamin A, vitamin E, sodium chloride, zinc chloride, polyvinylpyrrolidone and polysorbate 80.

8. The inhalable pharmaceutical composition of claim 7, wherein the dry powder comprises one or more pharmaceutically acceptable carriers and/or excipients selected from the group consisting of sodium citrate, sodium chloride, leucine or isoleucine and trehalose.

9. The inhalable pharmaceutical composition of claim 3, wherein the surfactant is polysorbate 80.

10. The inhalable pharmaceutical composition of claim 4, wherein microcrystalline particles have a specific surface area ranging from about 25 m²/g to about 63 m²/g.

11. The inhalable pharmaceutical dry powder composition of claim 4, wherein microcrystalline particles have a pore size ranging from about 23 nm to about 30 nm.

12. A method of treating idiopathic pulmonary fibrosis comprising:
administering to a patient in need of treatment by oral inhalation a dry powder composition comprising crystalline diketopiperazine particles and up to 50 mg of nintedanib or a pharmaceutically acceptable salt thereof, or Compound I of the formula: 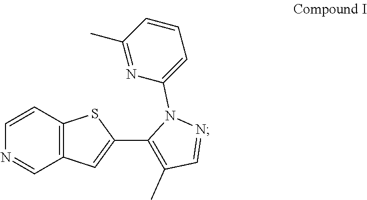 [SMILES: Cc1cccc(-n2ncc(C)c2-c2cc3cnccc3s2)n1]
or a pharmaceutically acceptable salt thereof;
and, optionally, one or more pharmaceutically acceptable carriers and/or excipients, wherein the dry powder composition is provided in a dry powder inhaler.

13. The method of claim 12, wherein a therapeutically effective dose of the dry powder composition is provided to said patient in one or more capsules or cartridges for adapting to said dry powder inhaler prior use and wherein each capsule or cartridge comprises up to 30 mg of nintedanib or Compound I.

14. The method of claim 13, wherein the therapeutically effective dose comprises up to 300 mg of nintedanib or Compound I per day provided in multiple cartridges.

15. The method of claim 12, wherein the one or more pharmaceutically acceptable carriers and/or excipients is selected from the group consisting of fumaryl diketopiperazine, lactose, mannose, sucrose, mannitol, trehalose, sodium citrate, trisodium citrate, zinc citrate, glycine, L-leucine, isoleucine, trileucine, sodium tartrate, zinc tartrate, methionine, vitamin A, vitamin E, sodium chloride, zinc chloride, polyvinylpyrrolidone, and polysorbate 80.

16. The method of claim 12, wherein the one or more pharmaceutically acceptable carriers and/or excipients is polysorbate 80.

17. The method of claim 12, wherein the diketopiperazine is fumaryl diketopiperazine.

18. The method of claim 12, wherein the dry powder composition is administered in at least one inhalation in less than 10 seconds per cartridge.

19. A method of treating idiopathic pulmonary fibrosis, comprising:
the inhalable pharmaceutical composition of claim 1, wherein the dry powder composition comprises about 2 mg, about 4 mg, about 6 mg, about 8 mg, or about 10 mg of nintedanib or a pharmaceutically acceptable salt thereof.

20. A dry powder inhaler comprising a movable member for mounting a cartridge and configuring a container to attain a dosing configuration, wherein said cartridge comprises the dry powder composition of claim 1.
